SEQUENCE LISTING (55 sequences)

SEQ ID NO: 1 - protein, 118 aa, synthetic construct
  REGION 1..118: note = Description of sequence: EphA3 scFv heavy chain amino acid sequence
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSS

SEQ ID NO: 2 - protein, 107 aa, synthetic construct
  REGION 1..107: note = Description of sequence: EphA3 scFv light chain amino acid sequence
DIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIK

SEQ ID NO: 3 - protein, 21 aa, synthetic construct
  REGION 1..21: note = Description of sequence: CD8 leader amino acid sequence
MALPVTALLLPLALLLHAARP

SEQ ID NO: 4 - protein, 15 aa, synthetic construct
  REGION 1..15: note = Description of sequence: Linker amino acid sequence
GGGGSGGGGSGGGGS

SEQ ID NO: 5 - protein, 45 aa, synthetic construct
  REGION 1..45: note = Description of sequence: CD8 hinge amino acid sequence
TTTPAPRPPTPAPTIASQPLSLRPEACRPAAGGAVHTRGLDFACD

SEQ ID NO: 6 - protein, 19 aa, synthetic construct
  REGION 1..19: note = Description of sequence: CD28 hinge amino acid sequence
LEPKSCDKTHTCPPCPDPK

SEQ ID NO: 7 - protein, 24 aa, synthetic construct
  REGION 1..24: note = Description of sequence: CD8 TM amino acid sequence
IYIWAPLAGTCGVLLLSLVITLYC

SEQ ID NO: 8 - protein, 27 aa, synthetic construct
  REGION 1..27: note = Description of sequence: CD28 TM amino acid sequence
FWVLVVVGGVLACYSLLVTVAFIIFWV

SEQ ID NO: 9 - protein, 42 aa, synthetic construct
  REGION 1..42: note = Description of sequence: 41BB amino acid sequence
KRGRKKLLYIFKQPFMRPVQTTQEEDGCSCRFPEEEEGGCEL

SEQ ID NO: 10 - protein, 41 aa, synthetic construct
  REGION 1..41: note = Description of sequence: CD28 amino acid sequence
RSKRSRLLHSDYMNMTPRRPGPTRKHYQPYAPPRDFAAYRS

SEQ ID NO: 11 - protein, 112 aa, synthetic construct
  REGION 1..112: note = Description of sequence: CD3z amino acid sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 484 aa, synthetic construct
  REGION 1..484: note = Description of sequence: EphA3 CAR K082 (H2L-BBz) amino acid sequence
MALPVTALLLPLALLLHAARPQVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSGGGGSGGGGSGGGGSDIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 460 aa, synthetic construct
  REGION 1..460: note = Description of sequence: EphA3 CAR K083 (H2L-28z) amino acid sequence
MALPVTALLLPLALLLHAARPQVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSGGGGSGGGGSGGGGSDIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 460 aa, synthetic construct
  REGION 1..460: note = Description of sequence: EphA3 CAR K084 (L2H-28z) amino acid sequence
MALPVTALLLPLALLLHAARPDIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKGGGGSGGGGSGGGGSQVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 484 aa, synthetic construct
  REGION 1..484: note = Description of sequence: EphA3 CAR K085 (L2H BBz) amino acid sequence
MALPVTALLLPLALLLHAARPDIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKGGGGSGGGGSGGGGSQVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 1452 bp, synthetic construct
  misc_feature 1..1452: note = Description of sequence: EphA3 CAR K082 (H2L-BBz)
  source 1..1452: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctccggtggaggtggttcgggaggtggaggtagcggaggtggtggatctgacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaaaccactacccctgcaccgcgaccaccaacaccggcgcccaccattgcgtcgcagcctctgtccctgcgcccagaagcatgccgtccagcagcaggtggtgcagttcatactcgtggtctggatttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 1380 bp, synthetic construct
  misc_feature 1..1380: note = Description of sequence: EphA3 CAR K083 (H2L-28z)
  source 1..1380: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctccggtggaggtggttcgggaggtggaggtagcggaggtggtggatctgacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaactcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - DNA, 1380 bp, synthetic construct
  misc_feature 1..1380: note = Description of sequence: EphA3 CAR K084 (L2H-28z)
  source 1..1380: mol_type = other DNA
atggccttaccagtgaccgccttgctcctgccgctggccttgctgctccacgccgccaggccggacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaaggtggaggtggttcgggaggtggaggtagcggaggtggtggatctcaggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctccctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 19 - DNA, 1452 bp, synthetic construct
  misc_feature 1..1452: note = Description of sequence: EphA3 CAR K085 (L2H BBz)
  source 1..1452: mol_type = other DNA
atggccttaccagtgaccgccttgctcctgccgctggccttgctgctccacgccgccaggccggacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaaggtggaggtggttcgggaggtggaggtagcggaggtggtggatctcaggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctccaccactacccctgcaccgcgaccaccaacaccggcgcccaccattgcgtcgcagcctctgtccctgcgcccagaagcatgccgtccagcagcaggtggtgcagttcatactcgtggtctggatttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 63 bp, synthetic construct
  misc_feature 1..63: note = Description of sequence: CD8 leader
  source 1..63: mol_type = other DNA
atggccttaccagtgaccgccttgctcctgccgctggccttgctgctccacgccgccaggccg

SEQ ID NO: 21 - DNA, 354 bp, synthetic construct
  misc_feature 1..354: note = Description of sequence: EphA3 scFv heavy chain
  source 1..354: mol_type = other DNA
caggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctcc

SEQ ID NO: 22 - DNA, 45 bp, synthetic construct
  misc_feature 1..45: note = Description of sequence: Linker
  source 1..45: mol_type = other DNA
ggtggaggtggttcgggaggtggaggtagcggaggtggtggatct

SEQ ID NO: 23 - DNA, 321 bp, synthetic construct
  misc_feature 1..321: note = Description of sequence: EphA3 scFv light chain
  source 1..321: mol_type = other DNA
gacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaa

SEQ ID NO: 24 - DNA, 135 bp, synthetic construct
  misc_feature 1..135: note = Description of sequence: CD8 hinge
  source 1..135: mol_type = other DNA
accactacccctgcaccgcgaccaccaacaccggcgcccaccattgcgtcgcagcctctgtccctgcgcccagaagcatgccgtccagcagcaggtggtgcagttcatactcgtggtctggatttcgcctgtgat

SEQ ID NO: 25 - DNA, 72 bp, synthetic construct
  misc_feature 1..72: note = Description of sequence: CD8 TM
  source 1..72: mol_type = other DNA
atctacatctgggcgcccttggccgggacttgtggggtccttctcctgtcactggttatcaccctttactgc

SEQ ID NO: 26 - DNA, 126 bp, synthetic construct
  misc_feature 1..126: note = Description of sequence: 41BB
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 27 - DNA, 57 bp, synthetic construct
  misc_feature 1..57: note = Description of sequence: CD28 hinge
  source 1..57: mol_type = other DNA
ctcgagcccaaatcttgtgacaaaactcacacatgcccaccgtgcccggatcccaaa

SEQ ID NO: 28 - DNA, 81 bp, synthetic construct
  misc_feature 1..81: note = Description of sequence: CD28 TM
  source 1..81: mol_type = other DNA
ttttgggtgctggtggtggttggtggagtcctggcttgctatagcttgctagtaacagtggcctttattattttctgggtg

SEQ ID NO: 29 - DNA, 123 bp, synthetic construct
  misc_feature 1..123: note = Description of sequence: CD28
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 30 - DNA, 336 bp, synthetic construct
  misc_feature 1..336: note = Description of sequence: CD3z
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - protein, 5 aa, synthetic construct
  REGION 1..5: note = Description of sequence: humanized anti-EGFRvIII binding domain VH CDR1
DYYIH

SEQ ID NO: 32 - protein, 17 aa, synthetic construct
  REGION 1..17: note = Description of sequence: humanized anti-EGFRvIII binding domain VH CDR2
RIDPENDETKYGPIFQG

SEQ ID NO: 33 - protein, 5 aa, synthetic construct
  REGION 1..5: note = Description of sequence: humanized anti-EGFRvIII binding domain VH CDR3
RGGVY

SEQ ID NO: 34 - protein, 16 aa, synthetic construct
  REGION 1..16: note = Description of sequence: humanized anti-EGFRvIII binding domain VL CDR1
KSSQSLLDSDGKTYLN

SEQ ID NO: 35 - protein, 7 aa, synthetic construct
  REGION 1..7: note = Description of sequence: humanized anti-EGFRvIII binding domain VL CDR2
LVSKLDS

SEQ ID NO: 36 - protein, 9 aa, synthetic construct
  REGION 1..9: note = Description of sequence: humanized anti-EGFRvIII binding domain VL CDR3
WQGTHFPGT

SEQ ID NO: 37 - protein, 246 aa, synthetic construct
  REGION 1..246: note = Description of sequence: humanized anti-EGFRvIII binding domain single chain variable fragment of monoclonal antibody clone 3C10
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 38 - protein, 24 aa, synthetic construct
  REGION 1..24: note = Description of sequence: humanized anti-EGFRvIII transmembrane domain
IYIWAPLAGTCGVLLLSLVITLYC

SEQ ID NO: 39 - protein, 45 aa, synthetic construct; identical to SEQ ID NO: 5
  REGION 1..45: note = Description of sequence: humanized anti-EGFRvIII hinge region

SEQ ID NO: 40 - protein, 56 aa, synthetic construct
  REGION 1..56: note = Description of sequence: CD3 zeta domain
RVKFSRSADAPAYKQGQNQLYNELNLGRREEYDVLDKRRGRDPEMGGKPRRKNPQE

SEQ ID NO: 41 - protein, 111 aa, synthetic construct
  REGION 1..111: note = Description of sequence: primary signaling domain
RVKFSRSADAPAYKQGQNQLYNELNLGRREEYDVLDKRRGRDPEMGGKPRRKNPQELYNELQKDKMAEAYSEIGMKGERRRGKGHDGLYQGLSTATKDTYDALHMQALPPR

SEQ ID NO: 42 - DNA, 1476 bp, synthetic construct
  misc_feature 1..1476: note = Description of sequence: human EGFRvIII-targeting chimeric antigen receptor 3C10-41BBz
  source 1..1476: mol_type = other DNA
atggccttaccagttaccgccttattattgcctttagccttattgttacatgccgcccgtccgggatccgagattcagctgcagcaatctggggcagaacttgtgaagccaggggcctcagtcaagctgtcctgcacaggttctggcttcaacattgaagactactatattcactgggtgaagcagaggactgaacagggcctggaatggattggaaggattgatcctgagaatgatgaaactaaatatggcccaatattccagggcagggccactataacagcagacacatcctccaacacagtctacctgcaactcagcagcctgacatctgaggacactgccgtctattactgtgcctttcgcggtggagtctactgggggccaggaaccactctcacagtctcctcaggaggtggtggttccggtggtggtggttccggaggtggtggttcacatatggatgttgtgatgacccagtctccactcactctatcggttgccattggacaatcagcctccatctcttgcaagtcaagtcagagcctcttagatagtgatggaaagacatatttgaattggttgttacagaggccaggccagtctccaaagcgcctaatctctctggtgtctaaactggactctggagtccctgacaggttcactggcagtggatcagggacagatttcacactgagaatcagcagagtggaggctgaggatttgggaatttattattgctggcaaggtacacattttcctgggacgttcggtggagggaccaagctggagataaaagctagcaccactacccctgcaccgcgaccaccaacaccggcgcccaccattgcgtcgcagcctctgtccctgcgcccagaagcatgccgtccagcagcaggtggtgcagttcatactcgtggtctggatttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 43 - protein, 491 aa, synthetic construct
  REGION 1..491: note = Description of sequence: human EGFRvIII-targeting chimeric antigen receptor 3C10-41BBz
MALPVTALLLPLALLLHAARPGSEIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSSGGGGSGGGGSGGGGSHMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 9 aa, synthetic construct
  REGION 1..9: note = Description of sequence: human EphA3 single chain variable fragment VH CDR3
GGYYEDFDS

SEQ ID NO: 45 - protein, 9 aa, synthetic construct
  REGION 1..9: note = Description of sequence: human EphA3 single chain variable fragment VL CDR3
GQYANYPYT

SEQ ID NO: 46 - protein, 118 aa, synthetic construct; identical to SEQ ID NO: 1
  REGION 1..118: note = Description of sequence: extracellular anti-human EphA3 binding domain VH

SEQ ID NO: 47 - protein, 107 aa, synthetic construct; identical to SEQ ID NO: 2
  REGION 1..107: note = Description of sequence: extracellular anti-human EphA3 binding domain VL

SEQ ID NO: 48 - protein, 5 aa, synthetic construct
  REGION 1..5: note = Description of sequence: extracellular anti-human EphA3 binding domain VH CDR1
SYWIN

SEQ ID NO: 49 - protein, 17 aa, synthetic construct
  REGION 1..17: note = Description of sequence: extracellular anti-human EphA3 binding domain VH CDR2
DIYPGSGNTNYDEKFKR

SEQ ID NO: 50 - protein, 9 aa, synthetic construct
  REGION 1..9: note = Description of sequence: extracellular anti-human EphA3 binding domain VH CDR3
SGYYEDFDS

SEQ ID NO: 51 - protein, 11 aa, synthetic construct
  REGION 1..11: note = Description of sequence: extracellular anti-human EphA3 binding domain VL CDR1
RASQEISGYLG

SEQ ID NO: 52 - protein, 7 aa, synthetic construct
  REGION 1..7: note = Description of sequence: extracellular anti-human EphA3 binding domain VL CDR2
AASTLDS

SEQ ID NO: 53 - protein, 9 aa, synthetic construct
  REGION 1..9: note = Description of sequence: extracellular anti-human EphA3 binding domain VL CDR3
VQYANYPYT

SEQ ID NO: 54 - DNA, 1183 bp, synthetic construct
  misc_feature 1..1183: note = Description of sequence: EF1 alpha promoter
  source 1..1183: mol_type = other DNA
gtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 55 - DNA, 1401 bp, synthetic construct
  misc_feature 1..1401: note = Description of sequence: CD8 leader, single chain variable fragment (scFv) of anti-human EphA3 monoclonal antibody ifabotuzumab (Ifab scFv) and CD8 hinge
  source 1..1401: mol_type = other DNA
gctagctctagaatggccttaccagtgaccgccttgctcctgccgctggccttgctgctccacgccgccaggccggacatccagatgacccagtctccatccttcctgtctgcatctgtaggagacagagtcaccatcacttgccgggccagtcagggcattatcagttatttagcctggtatcagcaaaaaccagagaaagcccctaagcgcctgatctatgctgcatccagtttgcaaagtggggtcccatcaaggttcagcggcagtggatctgggacagaattcactctcacaatcagcagcctgcagcctgaagattttgcaacttactactgcgggcagtatgccaattatccgtacacctttggccaaggtacgaaactggaaattaaaggtggaggtggttcgggaggtggaggtagcggaggtggtggatctcaggtgcagctggtgcagtctggggctgaggtgaagaagcctggggcctcagtgaaggtttcctgcaaggcttctggatacaccttcacaggctactggatgaattgggtgcgccaggcccccgggcaaggcctggagtggatgggggacatctacccgggcagtggtaacacaaactacgatgagaagttccagggtagagtcacgatgaccagggacacgtccatcagcacagcctacatggagctgagcaggctgagatctgacgacacagccgtgtactactgcgcaagaggtggatattatgaagattttgatagctggggccaaggtaccactgtgaccgtgagctccctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgac

(19) United States
(12) Patent Application Publication
(10) Pub. No.: US 2025/0171547 A1
(43) Pub. Date: May 29, 2025

(54) EPHA3 DIRECTED CAR-T CELLS FOR TREATMENT OF TUMORS

(71) Applicant: MAYO FOUNDATION FOR MEDICAL EDUCATION AND RESEARCH, Rochester, MN (US)

(72) Inventors: Cameron DURRANT, Oxford, FL (US); Dale CHAPPELL, Dolores, CO (US); Saad J. KENDERIAN, Rochester, MN (US)

(73) Assignee: MAYO FOUNDATION FOR MEDICAL EDUCATION AND RESEARCH, Rochester, MN (US)

(21) Appl. No.: 18/896,805

(22) Filed: Sep. 25, 2024

Related U.S. Application Data
(62) Division of application No. 17/093,370, filed on Nov. 9, 2020, now Pat. No. 12,139,544.
(60) Provisional application No. 62/933,354, filed on Nov. 8, 2019.

(51) Int. Cl. C07K 16/28; A61K 35/17; A61K 38/00; A61K 39/00; A61K 40/11; A61K 40/31; A61K 40/42; A61P 35/00; C07K 14/705; C07K 14/725; C07K 16/24; C12N 15/86

(52) U.S. Cl. CPC C07K 16/2866; A61K 35/17; A61K 40/11; A61K 40/31; A61K 40/4204; A61K 40/422; A61P 35/00; C07K 14/7051; C07K 14/70517; C07K 14/70521; C07K 14/70578; C07K 16/243; C07K 16/2818; C07K 16/2827; C12N 15/86; A61K 38/00; A61K 2039/507; A61K 2039/5156; A61K 2039/5158; A61K 2239/31; A61K 2239/38; A61K 2239/47; C07K 2317/24; C07K 2317/53; C07K 2317/622; C07K 2317/76; C07K 2319/02; C07K 2319/03; C07K 2319/30; C07K 2319/33; C12N 2740/15043

(57) ABSTRACT

This invention provides chimeric antigen receptors (CARs) targeting human EphA3 and dual targeting CARs that bind to human EphA3 and to human mutant epidermal growth factor receptor variant III (EGFRvIII). This invention also relates to CAR-T cells comprising the provided CARs or the dual targeting CARs. Methods for treating a solid tumor cancer by administering the CARs are provided.

### CROSS-REFERENCE TO RELATED APPLICATIONS

This application is a divisional application of U.S. application Ser. No. 17/093,370, filed Nov. 9, 2020, now allowed, which claims priority to U.S. Provisional Application No. 62/933,354, filed Nov. 8, 2019, each of which is hereby incorporated by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “P-589366-USD_ST26.xml”. The XML file is 86,771 bytes, created on Dec. 24, 2024, and is being submitted electronically via USPTO Patent Center.

### FIELD OF THE INVENTION

This invention relates to chimeric antigen receptors (CARs) targeting human EphA3 and binding thereto and to dual targeting CARs that bind to human EphA3 and to human mutant epidermal growth factor receptor variant III (EGFRvIII). This invention also relates to CAR-T cells comprising the CARs targeting human EphA3 and binding thereto and to CAR-T cells comprising the dual targeting CARs that bind to human EphA3 and to human EGFRvIII. This invention further relates to methods for treating a solid tumor cancer, the methods comprising administering to a subject in need thereof a pharmaceutical composition comprising the CAR-T cells comprising the CARs targeting human EphA3 and binding thereto or the CAR-T cells comprising the dual targeting CARs that bind to human EphA3 and to human EGFRvIII.

### BACKGROUND OF THE INVENTION

Efficacy of chimeric antigen receptor T cell (CART) therapy remains limited in solid tumors. Given the heterogeneity of surface receptor expression and immunosuppressive stromal microenvironment, strategies to target and disrupt tumor neovasculature and tumor stroma are needed to help overcome CART inhibition in solid tumors including glioblastoma multiforme (GBM). Eph receptors are the largest family of receptor tyrosine kinases and are integral to cell adhesion, migration, and axon guidance during development and homeostasis. EphA3 is a receptor tyrosine kinase which is poorly expressed in adult tissues but is highly expressed in tumor neovasculature and tumor stromal cells in GBM and other solid tumors. EphA3 is over-expressed in up to 40% of GBM samples and is over-expressed in the tumor stroma and tumor vasculature of other solid tumors including pancreatic, prostate, breast, colon, melanoma, myeloma, bladder, kidney and liver tumors, lung cancer, e.g., small cell lung cancer (SCLC) or non-small cell lung cancer (NSCLC).

Surgical removal of solid GBM tumors, temozolomide chemotherapy and radiation prolong survival by up to five years in about 7% of patients. Current therapies for GBM are not curative. In light of the inadequacies of surgery and current drugs and therapies for GBM and other solid tumors, there exists a critical need for improved compositions and therapeutically effective methods to treat solid tumor cancers, to reduce solid tumor cancer relapse rate or prevent occurrence of solid tumor cancer relapse in patients having or who have had a solid tumor cancer. The present invention provides CART cells directed against EphA3 to use in targeting tumor neovasculature and tumor stromal cells in solid tumors including GBM, pancreatic, prostate, breast, colon, melanoma, myeloma, lung, bladder, kidney and liver tumors.

### SUMMARY OF THE INVENTION

In one aspect, the present invention provides a chimeric antigen receptor (CAR) that binds to human Eph receptor A3 (EphA3), the CAR comprising an extracellular anti-human EphA3 binding domain comprising a single chain variable fragment (scFv) of anti-human EphA3 monoclonal antibody ifabotuzumab, wherein the human EphA3 scFv comprises a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2.

In another aspect, the present invention provides a CAR construct that binds to human EphA3 comprising: (a) a single chain variable fragment (scFv) of monoclonal antibody ifabotuzumab that binds to human EphA3, the scFv comprising a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2; wherein the V_{H} and the V_{L} are attached by a linker peptide comprising an amino acid sequence of SEQ ID NO: 4; (b) a hinge region comprising a CD8 hinge region comprises an amino acid sequence of SEQ ID NO: 5 or a CD28 hinge region comprises an amino acid sequence of SEQ ID NO: 6. (c) a transmembrane domain, and (d) an intracellular signaling domain comprising a costimulatory domain and an activation domain.

In an aspect, the present invention provides a method for producing a human EphA3-targeting CAR T-cell, the method comprising transducing a T-cell with the herein provided EphA3 CAR expression cassette.

In another aspect, the present invention provides methods for treating a solid tumor cancer, the methods comprising administering to a subject in need thereof a pharmaceutical composition comprising the herein provided human EphA3-targeting CAR-T cells.

In one aspect, the present invention provides a dual targeting CAR-T cell that binds to human EphA3 and to human mutant epidermal growth factor receptor variant III (EGFRvIII), the CAR-T cell comprising: a first CAR construct that binds to human Eph receptor A3 (EphA3), the CAR comprising an extracellular anti-human EphA3 binding domain comprising a human EphA3 single chain variable fragment (scFv) of anti-EphA3 monoclonal antibody ifabotuzumab, wherein the human EphA3 scFv comprises a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2, wherein the scFv of monoclonal antibody ifabotuzumab that binds to human EphA3, is fused in tandem to a hinge region, a transmembrane domain; and an intracellular signaling domain comprising a costimulatory domain and a activation domain; and a second CAR construct that binds to human mutant epidermal growth factor receptor variant III (EGFRvIII) comprising a humanized anti-EGFRvIII binding domain, wherein the humanized anti-EGFRvIII binding domain comprises: (a) a heavy chain immunoglobulin variable region comprising: (i) a CDR1 comprising amino acid sequence DYYIH (SEQ ID NO: 31); (ii) a CDR2 comprising amino acid sequence RIDPENDETKYGPIFQG (SEQ ID NO: 32); and (iii) a CDR3 comprising amino acid sequence RGGVY (SEQ ID NO: 33); and (b) a light chain immunoglobulin variable region comprising: (i) a CDR1 comprising amino acid sequence KSSQSLLDSDGKTYLN (SEQ ID NO: 34); (ii) a CDR2 comprising the sequence LVSKLDS (SEQ ID NO: 35); and (iii) a CDR3 comprising amino acid sequence WQGTHFPGT (SEQ ID NO: 36).

In another aspect, the present invention provides a method for producing a dual targeting CAR-T cell that binds to human EphA3 and human EGFRvIII, the method comprising: (a) transducing a T-cell with an EphA3-targeting CAR expression cassette comprising a CAR that binds to human EphA3 and is encoded by an isolated nucleic acid sequence of SEQ ID NO: 16, SEQ ID NO: 17, SEQ ID NO: 18 or SEQ ID NO: 19 to produce a human EphA3-targeting CAR T-cell; and (b) transducing the human EphA3-targeting CAR T-cell produced in step (a) with an expression cassette comprising a human EGFRvIII-targeting CAR that binds to human EGFRvIII and the human EGFRvIII-targeting CAR is encoded by a nucleic acid sequence of SEQ ID NO: 42:

ATGGCCTTACCAGTTACCGCCTTATTATTGCCTTTAGCCTTATTGTTAC
ATGCCGCCCGTCCGGGATCCGAGATTCAGCTGCAGCAATCTGGGGCAGA
ACTTGTGAAGCCAGGGGCCTCAGTCAAGCTGTCCTGCACAGGTTCTGGC
TTCAACATTGAAGACTACTATATTCACTGGGTGAAGCAGAGGACTGAAC
AGGGCCTGGAATGGATTGGAAGGATTGATCCTGAGAATGATGAAACTAA
ATATGGCCCAATATTCCAGGGCAGGGCCACTATAACAGCAGACACATCC
TCCAACACAGTCTACCTGCAACTCAGCAGCCTGACATCTGAGGACACTG
CCGTCTATTACTGTGCCTTTCGCGGTGGAGTCTACTGGGGGCCAGGAAC
CACTCTCACAGTCTCCTCAGGAGGTGGTGGTTCCGGTGGTGGTGGTTCC
GGAGGTGGTGGTTCACATATGGATGTTGTGATGACCCAGTCTCCACTCA
CTCTATCGGTTGCCATTGGACAATCAGCCTCCATCTCTTGCAAGTCAAG
TCAGAGCCTCTTAGATAGTGATGGAAAGACATATTTGAATTGGTTGTTA
CAGAGGCCAGGCCAGTCTCCAAAGCGCCTAATCTCTCTGGTGTCTAAAC
TGGACTCTGGAGTCCCTGACAGGTTCACTGGCAGTGGATCAGGGACAGA
TTTCACACTGAGAATCAGCAGAGTGGAGGCTGAGGATTTGGGAATTTAT
TATTGCTGGCAAGGTACACATTTTCCTGGGACGTTCGGTGGAGGGACCA
AGCTGGAGATAAAAGCTAGCACCACTACCCCTGCACCGCGACCACCAAC
ACCGGCGCCCACCATTGCGTCGCAGCCTCTGTCCCTGCGCCCAGAAGCA
TGCCGTCCAGCAGCAGGTGGTGCAGTTCATACTCGTGGTCTGGATTTCG
CCTGTGATATCTACATCTGGGCGCCCTTGGCCGGGACTTGTGGGGTCCT
TCTCCTGTCACTGGTTATCACCCTTTACTGCAAACGGGGCAGAAAGAAA
CTCCTGTATATATTCAAACAACCATTTATGAGACCAGTACAAACTACTC
AAGAGGAAGATGGCTGTAGCTGCCGATTTCCAGAAGAAGAAGAAGGAGG
ATGTGAACTGAGAGTGAAGTTCAGCAGGAGCGCAGACGCCCCCGCGTAC
AAGCAGGGCCAGAACCAGCTCTATAACGAGCTCAATCTAGGACGAAGAG
AGGAGTACGATGTTTTGGACAAGAGACGTGGCCGGGACCCTGAGATGGG
GGGAAAGCCGAGAAGGAAGAACCCTCAGGAAGGCCTGTACAATGAACTG
CAGAAAGATAAGATGGCGGAGGCCTACAGTGAGATTGGGATGAAAGGCG
AGCGCCGGAGGGGCAAGGGGCACGATGGCCTTTACCAGGGTCTCAGTAC
AGCCACCAAGGACACCTACGACGCCCTTCACATGCAGGCCCTGCCCCCT
CGCTAA

to produce a human EphA3-targeting- and a human EGFRvIII-targeting-CAR T-cell, wherein the human EphA3-targeting- and human EGFRvIII-targeting-CAR T-cell expresses the human EphA3-targeting CAR and the human EGFRvIII-targeting CAR on a cell surface thereof.

In additional aspects, the present invention provides a pharmaceutical composition comprising the herein provided dual targeting CAR-T cell that binds to human EphA3 and to human mutant EGFRvIII and a pharmaceutically acceptable carrier.

In additional aspects, the present invention provides a pharmaceutical composition comprising a CAR-T cell that binds to human EphA3 and a pharmaceutical composition comprising CAR-T cell that binds to human mutant EGFRvIII, wherein each pharmaceutical composition is administered to a subject in need thereof together (concurrently) or sequentially.

In other aspects, the present invention provides a chimeric antigen receptor (CAR) construct that binds to human Eph receptor A3 (EphA3), the CAR comprising an extracellular anti-human EphA3 binding domain comprising a human EphA3 single chain variable fragment (scFv) of anti-EphA3 monoclonal antibody, wherein the human EphA3 scFv comprises CDR3 of the V_{H} region comprises GGYYEDFDS (SEQ ID NO:44) and the CDR3 of the V_{L} region comprises GQYANYPYT (SEQ ID NO:45).

In another aspect, the present invention provides a CAR construct that binds to human Eph receptor A3 (EphA3), the CAR construct comprising an extracellular anti-human EphA3 binding domain comprising: a V_{H} region comprising amino acid sequence
QVQLVQSGAEVKKPGASVKVSCKASGYTFTGYWMNWVRQAPGQGLEWMGD IYPGSGNTNYDEKFQGRVTMTRDTSISTAYMELSRLRSDDTAVYYCARGG YYEDFDSWGQGTTVTVSS (SEQ ID NO:46) and
a V_{L} region comprising amino acid sequence
DIQMTQSPSFLSASVGDRVTITCRASQGIISYLAWYQQKPEKAPKRLIYA ASSLQSGVPSRFSGSGSGTEFTLTISSLQPEDFATYYCGQYANYPYTFGQ GTKLEIK (SEQ ID NO:47).

In one aspect, the present invention provides a CAR construct that binds to human Eph receptor A3 (EphA3), the CAR construct comprising an extracellular anti-human EphA3 binding domain comprising: a V_{H} region CDR1 having a sequence SYWIN (SEQ ID NO:48), a V_{H} region CDR2 having a sequence DIYPGSGNTNYDEKFKR (SEQ ID NO:49), a V_{H} region CDR3 having a sequence SGYYEDFDS (SEQ ID NO:50), and a V_{L} region CDR1 having a sequence RASQEISGYLG (SEQ ID NO:51), a V_{L} region CDR2 having a sequence AASTLDS (SEQ ID NO:52), and a V_{L} region CDR3 having a sequence VQYANYPYT (SEQ ID NO:53).

Other features and advantages of the present invention will become apparent from the following detailed description, examples and figures. It should be understood, however, that the detailed description and the specific examples while indicating certain embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1C show CAR-T cell production of the Epha-3 targeting CAR (termed K84) on the surface of donor T-cells by flow cytometry (FIGS. 1A-1B) and a map of the EphA-3 targeting CAR construct termed K84 (FIG. 1C).

FIGS. 2A-2B show GBM6 killing by the EphA-3 targeting CAR (termed K84) in a 24 hour TdT killing assay compared to un-transduced (UTD) cells from the same donor apheresis cone. The GBM6 EphA-3 expression was 19.28%. The CAR 84 demonstrated significant killing of GBM6 over 24 hours compared to UTD.

FIGS. 3A-3B show the killing of GBM PDX line 76 (“GBM 76”) by EphA-3 targeting CAR (termed K84) in a 24 hour killing assay. The GBM 76 Epha-3 expression was 25.05% RNA expression. The CAR 84 demonstrated significant killing of GBM 76 over 24 hours compared to UTD.

FIGS. 4A-4B show the killing of GBM PDX line 39 (“GBM 39”) by EphA-3 targeting CAR (termed K84) in a 24 hour killing assay. There was not a significant difference in killing when co-cultured with cell lines with 6.95% EphA3 expression (FIG. 4A).

FIGS. 5A-5D show CAR-T cell production of the EGFRviii targeting CAR (termed K96 or K096) on the surface of donor T-cells via flow cytometry (FIGS. 5A-5C) and a map of the EGFRviii targeting CAR construct termed K96 (FIG. 5D).

FIGS. 6A-6B show the killing of GBM PDX line 39 (“GBM 39”) by EGFRviii targeting CAR (termed K96) in a 24 hour TdT killing assay. The CAR K96 demonstrated significant killing of GBM 39 over 24 hours (from low effector:target ratio of 1.25:1) compared to UTD.

FIGS. 7A-7B show the killing of GBM PDX line 39 (“GBM 39”) by EGFRviii targeting CAR (termed K96) in a 48 hour TdT killing assay. The CAR K96 demonstrated significant killing of GBM 39 over 48 hours (from low effector:target ratio of 0.625:1) compared to UTD.

FIGS. 8A-8C show a proliferation assay for K96 EGFRviii targeting CAR-T cells (EGFRviii CART) in a 5 day incubation versus radiated cell lines for GBM 39 (FIG. 8A), GBM 76 (FIG. 8B) and GBM 22 (FIG. 8C) compare to UTD.

FIGS. 9A-9B show the killing of GBM PDX line 39 (“GBM 39”) by Epha-3 targeting CAR (termed K84) in a 48 hour TdT killing assay. The GBM 39 EphA-3 expression was 6.95% RNA expression.

FIGS. 10A-10B show the killing of GBM PDX line 39 (“GBM 39”) by EGFRviii targeting CAR (termed K96) in a 48 hour TdT killing assay. The CAR K96 demonstrated significant killing of GBM 39 over 48 hours (from low effector:target ratio of 0.625:1) compared to UTD.

FIG. 11 shows the killing over 24 hours of GBM 76 by K84 (comprising EphA3 targeting CAR cloned into lentivirus), K96 (comprising EGFRviii targeting CAR cloned into lentivirus), “both”=K84+K96” (i.e., CAR T cells were exposed to and cultured with one lentivirus, e.g., K84, and then mixed with CAR T cells exposed to and cultured with the other lentivirus, e.g., K96) and “combinatorial CAR T cells” exposed to and cultured with both lentiviruses K84 and K96 compared to UTD. EGFRviii expression was present in this GBM line and Epha-3 RNA expression was 25.05%. The terms “both”=“K84+K96” and “combinatorial CAR T cells” (also called “combi”) are used herein throughout as described for FIG. 11.

FIG. 12 shows the killing (in a TdT assay) over 72 hours of GBM 76 by K84, K96, “both K84+K96” and “combinatorial CAR T cells” compared to UTD. GBM 76 EphA3 expression was 25.02%.

FIG. 13 shows the killing (in a TdT assay) over 72 hours of GBM 76 by K84, K96, “both K84+K96” and “combinatorial CAR T cells” compared to UTD. GBM 76 EphA3 expression was 25.02%.

FIG. 14 shows the killing over 72 hours of GBM 108 by K84, K96, “both K84+K96” and “combinatorial CAR T cells” compared to UTD. GBM 76 EphA3 expression was 25.02% compared to UTD. EGFRviii expression was present in this GBM line.

FIGS. 15A-15B show the killing over 7 days incubation of GBM 76 by K84, K96, “both K84+K96” and “combinatorial CAR T cells” compared to UTD. GBM 76 EphA3 expression was 25.02% RNA expression.

FIGS. 16A-16B show the killing over 7 days incubation of GBM 108 by K84, K96, “both K84+K96” and “combinatorial CAR T cells” compared to UTD. This GBM line showed EphA3 expression of 1.52% RNA expression and EGFRviii expression was present.

FIG. 17 shows GBM 6 killing assays over 5 days incubation by CAR T cells: K84, K96, “both K84+K96” and “combinatorial CAR T cells” compared to UTD. GBM 6 EphA3 showed 19.28% expression per RNA seq; this GBM does have (expresses) EGFR and EGFRviii.

FIG. 18 shows a duplicate killing assay of GBM 6 over 5 days incubation by CAR T cells: K96 and “combinatorial CAR T cells” compared to UTD. Killing assays with K84 or both K84+K96 were not performed.

FIG. 19 shows a proliferation assay for K96 (EGFRviii targeting CAR-T cells) and “combinatorial CAR T cells” for GBM 6 line compared to UTD.

FIG. 20 shows a proliferation assay for K96 (EGFRviii targeting CAR-T cells) and “combinatorial CAR T cells” for GBM 150 line compared to UTD. GBM 150 is not supposed to have any EGFRviii expression, but it does have three copies of EGFR (chromosome 7). GBM 150 has 96.33% EphA3 expression per RNA seq.

FIG. 21 shows a killing assay of GBM 6 over 20 days incubation by K84, K96, “both K84+K96” and “combinatorial CAR T cells” compared to UTD.

FIG. 22 shows a killing assay of GBM 6 over 20 days incubation by K96 and “combinatorial CAR T cells” compared to UTD. A one way ANOVA was performed comparing UTD versus K96 and UTD versus combinatorial (p value is for both tests).

FIGS. 23A-23C show binding of IIIA4 antibody to NCI-H446 Cell line (Human SCLC).

FIG. 24 shows the CAR-T cells for solid tumors in ongoing trials or non-active trials adapted from Hartman et al. EMBO Mol Med. 2017 September; 9(9):1183-1197.

The human EphA3-targeting CAR-T cells described herein are administered (via a pharmaceutical composition comprising the human EphA3-targeting CAR-T cells) as either an adjuvant or in combination with two or more pharmaceutical compositions comprising the CAR-T cells for solid tumors in FIG. 24.

FIG. 25 shows EphA3 CAR expression on transduced T-cells (right) compared to untransduced T-cells (Left). Construct map (bottom).

FIGS. 26A-26C show EphA3 CAR bioluminescence killing assay at 24 hour incubation versus GBM 76, GBM 6 and GBM 39.

FIGS. 27A-27B show dual EphA3/EGFRvIII targeting CART cells exhibit potent antitumor killing against patient derived GBM.

FIG. 28 shows combinatorial EGFRvIII and EphA3 CARs demonstrate increased proliferation when exposed to target antigen.

### DETAILED DESCRIPTION OF THE INVENTION

The present invention may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

GBM is the most aggressive adult brain cancer. It is characterized by highly infiltrative and heterogeneous cells. Despite surgical removal of a GBM tumor, most patients with GBM develop recurrent tumors near the original tumor site or at distant locations in the brain.

Studies have implicated overexpression of the receptor tyrosine kinase Eph receptor A3 (EphA3) in glioblastoma (GBM). EphA3 also has been shown to be elevated in various hematological cancers and solid tumors. It has been reported that EphA3 has oncogenic functions in GBM. In particular, EphA3 has been found to be predominantly expressed on glioma stem cells (GSCs), while it is expressed in low levels in normal brain tissue.

The present invention is directed to the treatment of GBM and other solid tumors overexpressing EphA3 by administration of a chimeric antigen receptor that binds to human EphA3 on tumor neovasculature and tumor stromal cells.

Unless otherwise defined herein, scientific, and technical terms used in connection with this application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

In one aspect, the present invention provides a chimeric antigen receptor (CAR) that binds to human Eph receptor A3 (EphA3), the CAR comprising an extracellular anti-human EphA3 binding domain comprising a single chain variable fragment (scFv) of anti-human EphA3 monoclonal antibody ifabotuzumab, wherein the human EphA3 scFv comprises a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2. In an embodiment, the V_{H} and the V_{L} are attached by a linker peptide. In another embodiment, the linker peptide comprises an amino acid sequence of SEQ ID NO: 4. In an embodiment, the extracellular anti-human EphA3 binding domain is connected to a transmembrane domain by a hinge region. In some embodiments, the hinge region comprises a CD8 hinge region or a CD28 hinge region. In another embodiment, the CD8 hinge region comprises an amino acid sequence of SEQ ID NO: 5. In some embodiments, the CD28 hinge region comprises an amino acid sequence of SEQ ID NO: 6. In an embodiment, when the hinge region comprises a CD8 hinge region the transmembrane domain comprises a CD8 transmembrane domain. In another embodiment, when the hinge region comprises a CD28 hinge region the transmembrane domain comprises a CD28 transmembrane domain. In an embodiment, the CD8 transmembrane domain comprises an amino acid sequence of SEQ ID NO: 7. In another embodiment, the CD28 transmembrane domain comprises an amino acid sequence of SEQ ID NO: 8. In an embodiment of the herein provided CAR, the CAR further comprises a leader sequence. In some embodiments, the leader sequence comprises an amino acid sequence of SEQ ID NO: 3. In an embodiment, the CAR further comprises an intracellular signaling domain. In another embodiment, the intracellular signaling domain comprises a costimulatory domain and an activation domain. In some embodiments of the provided CAR, the costimulatory domain comprises a 4-1BB costimulatory domain or a CD28 costimulatory domain. In another embodiment, the activation domain is a CD3 zeta (CD3z or CD3ζ) activation domain. In an embodiment, the 4-1BB costimulatory domain costimulatory domain comprises an amino acid sequence of SEQ ID NO: 9. In a particular embodiment, the CD28 costimulatory domain comprises an amino acid sequence of SEQ ID NO: 10. In another embodiment, the CD3z activation domain comprises an amino acid sequence of SEQ ID NO: 11.

In another aspect, the present invention provides a CAR construct that binds to human EphA3 comprising: (a) a single chain variable fragment (scFv) of monoclonal antibody ifabotuzumab that binds to human EphA3, the scFv comprising a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2; wherein the V_{H} and the V_{L} are attached by a linker peptide comprising an amino acid sequence of SEQ ID NO: 4; (b) a hinge region comprising a CD8 hinge region comprises an amino acid sequence of SEQ ID NO: 5 or a CD28 hinge region comprises an amino acid sequence of SEQ ID NO: 6. (c) a transmembrane domain, and (d) an intracellular signaling domain comprising a costimulatory domain and an activation domain.

In an embodiment of the CAR construct that binds to human EphA3, the hinge region comprises the CD8 hinge region, the CD8 hinge region comprises an amino acid sequence of SEQ ID NO: 5. In another embodiment, wherein when the hinge region comprises a CD28 hinge region, the CD28 hinge region comprises an amino acid sequence of SEQ ID NO: 6. In some embodiments, the CAR construct further comprises a CD8 transmembrane domain comprising an amino acid sequence of SEQ ID NO: 7. In an embodiment, the CAR construct further comprises a CD28 transmembrane domain comprising an amino acid sequence of SEQ ID NO: 8. In a particular embodiment, the costimulatory domain comprises a 4-1BB costimulatory domain or a CD28 costimulatory domain. In another embodiment, the activation domain is a CD3z activation domain. In some embodiments, the 4-1BB costimulatory domain costimulatory domain comprises an amino acid sequence of SEQ ID NO: 9. In an embodiment, the CD28 costimulatory domain comprises an amino acid sequence of SEQ ID NO: 10. In some embodiments, the CD3z activation domain comprises an amino acid sequence of SEQ ID NO: 11. In a particular embodiment, the scFv of monoclonal antibody ifabotuzumab that binds to human EphA3, the hinge region, the transmembrane domain, and the intracellular signaling domain are fused in tandem.

In an embodiment, the human EphA3-targeting CAR expression cassette comprising the second generation CAR is cloned into a lentivirus backbone under control of a promoter, as described herein. In another embodiment of the human EphA3-targeting CAR expression cassette of claim 32, the promoter is human elongation factor-1 alpha (EF-1α). In an embodiment, the EF1 alpha promoter has a nucleic acid of (SEQ ID NO: 54):

GTGAGGCTCCGGTGCCCGTCAGTGGGCAGAGCGCACATCGCCCACAGTCC
CCGAGAAGTTGGGGGGAGGGGTCGGCAATTGAACCGGTGCCTAGAGAAGG
TGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTGT
GGTTCCCGCGGGCCTGGCCTCTTTACGGGTTATGGCCCTTGCGTGCCTTG
AATTACTTCCACCTGGCTGCAGTACGTGATTCTTGATCCCGAGCTTCGGG
TTGGAAGTGGGTGGGAGAGTTCGAGGCCTTGCGCTTAAGGAGCCCCTTCG
CCTCGTGCTTGAGTTGAGGCCTGGCCTGGGCGCTGGGGCCGCCGCGTGCG
AATCTGGTGGCACCTTCGCGCCTGTCTCGCTGCTTTCGATAAGTCTCTAG
CCATTTAAAATTTTTGATGACCTGCTGCGACGCTTTTTTTCTGGCAAGAT
AGTCTTGTAAATGCGGGCCAAGATCTGCACACTGGTATTTCGGTTTTTGG
GGCCGCGGGCGGCGACGGGGCCCGTGCGTCCCAGCGCACATGTTCGGCGA
GGCGGGGCCTGCGAGCGCGGCCACCGAGAATCGGACGGGGGTAGTCTCAA
GCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGG
CGCTCGGGAGAGCGGGCGGGTGAGTCACCCACACAAAGGAAAAGGGCCTT
TCCGTCCTCAGCCGTCGCTTCATGTGACTCCACGGAGTACCGGGCGCCGT
CCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGA
GACTGAAGTTAGGCCAGCTTGGCACTTGATGTAATTCTCCTTGGAATTTG
CCCTTTTTGAGTTTGGATCTTGGTTCATTCTCAAGCCTCAGACAGTGGTT
CAAAGTTTTTTTCTTCCATTTCAGGTGTCGTGA.

In some embodiments of the human EphA3-targeting CAR expression cassette, the lentivirus backbone is a third generation wherein the lentivirus backbone. In an embodiment, the human EphA3-targeting CAR T-cell comprising the herein provided EphA3-targeting CAR expression cassette, wherein the CAR-T cell expresses the CAR on a cell surface thereof, as described herein. In another embodiment, the EphA3-targeting CAR T-cell is an autologous EphA3-targeting CAR T-cell.

In an embodiment, provided herein is a pharmaceutical composition comprising the human EphA3-targeting CAR-T cell, as described herein, and a pharmaceutically acceptable carrier. In some embodiments of the pharmaceutical composition, the human EphA3-targeting CAR-T cell is autologous.

In an embodiment, provided herein is a method for producing a human EphA3-targeting CAR T-cell, the method comprising transducing a T-cell with the EphA3 CAR expression cassette, as described herein.

In a further embodiment, provided herein is a method for treating a solid tumor cancer, the method comprising administering to a subject in need thereof the pharmaceutical compositions described herein. In a particular embodiment, the solid tumor cancer is a brain cancer. In a specific embodiment, the brain cancer is glioblastoma multiforme (GBM). In an embodiment, the solid tumor cancer is a colon, kidney, bladder, breast, liver, pancreatic, prostate tumor, a melanoma, myeloma, or a lung cancer, e.g., small cell lung cancer (SCLC) or non-small cell lung cancer (NSCLC). In another embodiment, the solid tumor cancer expresses EphA3 on a surface of tumor cells, tumor-associated vasculature and/or tumor stroma. In an embodiment, the tumor stroma cells comprise myeloid derived suppressor cells. In an embodiment, the tumor cells comprise tumor stem cells. In particular embodiments, the tumor-associated vasculature comprises neo-vasculature.

In an embodiment, the method further comprises administering a human GM-CSF (hGM-CSF) antagonist selected from the group consisting of an anti-human GM-CSF antibody, an anti-hGM-CSF antibody fragment, a soluble hGM-CSF receptor alpha, an anti-hGM-CSF receptor (GM-CSFr) antibody and an anti-hGM-CSFr antibody fragment. In a specific embodiment, the anti-human GM-CSF antibody is hGM-CSF neutralizing antibody lenzilumab. In a specific embodiment the anti-human GM-CSF antibody is Namilumab, Otilimab, Gimsilumab, and TJM2 (TJ003234). In a further embodiment, the GM-CSF antagonist is anti-GM-CSF receptor antibody Mavrilimumab.

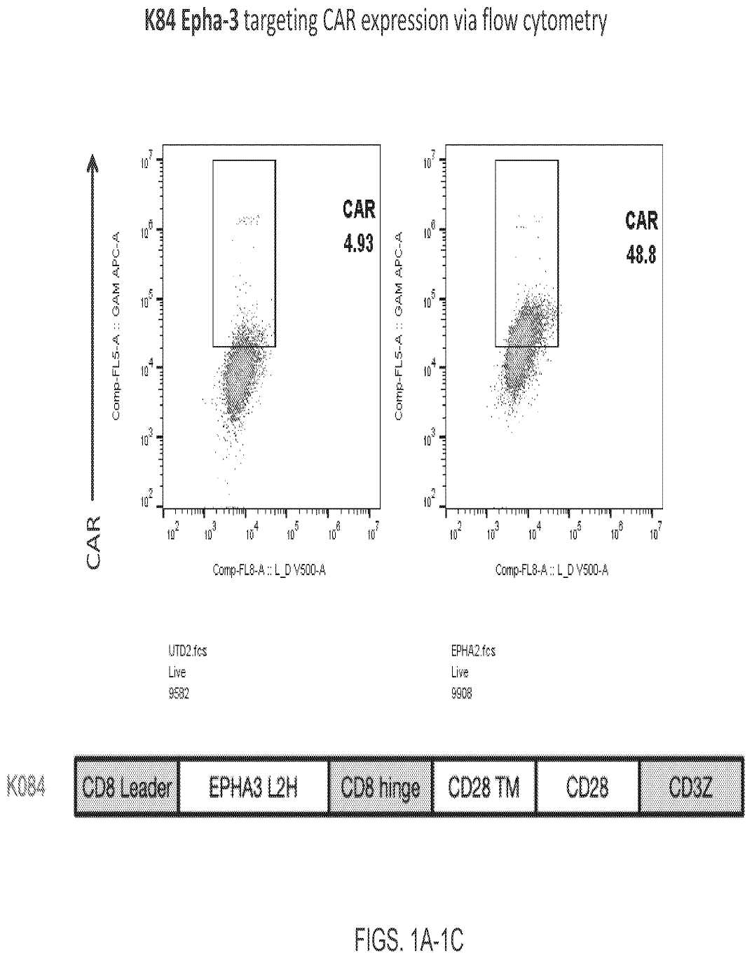

An anti-human GM-CSF antibody administered according to the invention may comprise any of the V_{H} regions VH #1, VH #2, VH #3, VH #4, or VH #5 as shown in FIG. 1 of U.S. Pat. Nos. 8,168,183 and 9,017,674, each of which is incorporated herein by reference in its entirety. In a particular embodiment, the anti-human GM-CSF antibody comprises VH #5 and VLK #2, as shown in FIG. 1 of U.S. Pat. Nos. 8,168,183 and 9,017,674, each of which is incorporated herein by reference in its entirety.

In some embodiments, an anti-human GM-CSF antibody of the invention may comprise any of the V_{L} regions VK #1, VK #2, VK #3, or VK #4 as shown in FIG. 1. In some embodiments, the anti-human GM-CSF antibody has a V_{H} region VH #1, VH #2, VH #3, VH #4, or VH #5 as shown in FIG. 1 of U.S. Pat. Nos. 8,168,183 and 9,017,674, each of which is incorporated herein by reference in its entirety; and a V_{L} region VK #1, VK #2, VK #3, or VK #4 as shown in FIG. 1, as described, e.g., in U.S. Pat. Nos. 8,168,183 and 9,017,674, each of which is incorporated herein by reference in its entirety.

In an embodiment, administration of lenzilumab reduces relapse rate or prevents occurrence of solid tumor relapse. In another embodiment, administration of lenzilumab prevents or reduces incidence of immunotherapy-related toxicity in the subject. In some embodiments, the immunotherapy-related toxicity is EphA3-targeting CAR-T cell related toxicity. In an embodiment, the EphA3-targeting CAR-T cell related toxicity is cytokine release syndrome, neurotoxicity and/or neuro-inflammation. In another embodiment, the method further comprises administering GM-CSF silenced EphA3-targeting CAR-T cells or gene knockout (GM-CSF^{k/o}) EphA3-targeting CAR-T cells, wherein administration of GM-CSF silenced EphA3-targeting CAR-T cells or GM-CSF^{k/o} EphA3-targeting CAR-T cells prevents or reduces incidence of immunotherapy-related toxicity in the subject. In an embodiment, administration of the GM-CSF silenced EphA3-targeting CAR-T cells or GM-CSF^{k/o} EphA3-targeting CAR-T cells reduces relapse rate or prevents occurrence of solid tumor relapse. In another embodiment, the method further comprises administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody. In an embodiment, the method further comprises administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody.

In a specific embodiment, the anti-programmed cell death-1 antibody is Pembrolizumab. In another embodiment the anti-programmed death-ligand antibody is avelumab. In another embodiment the anti-cytotoxic T-lymphocyte associated protein 4 (CTLA-4) antibody is Ipilimumab.

In another embodiment, the present invention provides a method for reducing solid tumor cancer relapse rate or preventing occurrence of solid tumor cancer relapse in a subject in need thereof, the method comprising administering to a subject a pharmaceutical composition, as described herein. In one embodiment, the solid tumor cancer is a brain cancer. In another embodiment, the brain cancer is glioblastoma multiforme (GBM). In an embodiment, the solid tumor cancer is a colon, kidney, bladder, breast, liver, pancreatic, prostate, melanoma, myeloma, lung, including non-small cell lung cancer (NSCLC) or a small cell lung cancer (SCLC). In another embodiment, the solid tumor cancer expresses EphA3 on a surface of tumor cells, tumor-associated vasculature and/or tumor stroma. In an embodiment, the tumor stroma cells comprise myeloid derived suppressor cells. In one embodiment, the tumor cells comprise tumor stem cells. In another embodiment, the tumor-associated vasculature comprises neo-vasculature. In still another embodiment, the method further comprises administering a human GM-CSF (hGM-CSF) antagonist selected from the group consisting of an anti-human GM-CSF antibody, an anti-hGM-CSF antibody fragment, a soluble hGM-CSF receptor alpha, an anti-hGM-CSF receptor (GM-CSFr) antibody and an anti-hGM-CSFr antibody fragment. In an embodiment, the anti-human GM-CSF antibody is hGM-CSF neutralizing antibody lenzilumab. In a specific embodiment the anti-human GM-CSF antibody is Namilumab, Otilimab, Gimsilumab, and TJM2 (TJ003234). In a further embodiment, the GM-CSF antagonist is anti-GM-CSF receptor antibody Mavrilimumab. In another embodiment, administration of lenzilumab reduces relapse rate or prevents occurrence of solid tumor relapse. In some embodiments, administration of lenzilumab prevents or reduces incidence of immunotherapy-related toxicity in the subject. In an embodiment, the immunotherapy-related toxicity is EphA3-targeting CAR-T cell related toxicity. In another embodiment, the EphA3-targeting CAR-T cell related toxicity is cytokine release syndrome, neurotoxicity and/or neuro-inflammation. In an embodiment, the method further comprises administering GM-CSF silenced EphA3-targeting CAR-T cells or gene knockout (GM-CSF^{k/o}) EphA3-targeting CAR-T cells, wherein administration of GM-CSF silenced EphA3-targeting CAR-T cells or GM-CSF^{k/o} EphA3-targeting CAR-T cells prevents or reduces the incidence of immunotherapy-related toxicity in the subject. In an embodiment, the method further comprises administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody. In a specific embodiment, the anti-programmed cell death-1 antibody is Pembrolizumab. In another embodiment the anti-programmed death-ligand antibody is avelumab. In another embodiment the anti-cytotoxic T-lymphocyte associated protein 4 (CTLA-4) antibody is Ipilimumab

In one aspect, the present invention provides a dual targeting CAR-T cell that binds to human EphA3 and to human mutant epidermal growth factor receptor variant III (EGFRvIII), the CAR-T cell comprising:
a first CAR construct that binds to human Eph receptor A3 (EphA3), the CAR comprising an extracellular anti-human EphA3 binding domain comprising a human EphA3 single chain variable fragment (scFv) of anti-EphA3 monoclonal antibody ifabotuzumab, wherein the human EphA3 scFv comprises a heavy chain immunoglobulin variable region (V_{H}) comprising an amino acid sequence of SEQ ID NO: 1 and a light chain immunoglobulin variable region (V_{L}) comprising an amino acid sequence of SEQ ID NO: 2.
   wherein the scFv of monoclonal antibody ifabotuzumab that binds to human EphA3, is fused in tandem to a hinge region, a transmembrane domain; and an intracellular signaling domain comprising a costimulatory domain and an activation domain;
   and
a second CAR construct that binds to human mutant epidermal growth factor receptor variant III (EGFRvIII) comprising a humanized anti-EGFRvIII binding domain, wherein the humanized anti-EGFRvIII binding domain comprises:
   (a) a heavy chain immunoglobulin variable region comprising:
      (i) a CDR1 comprising amino acid sequence DYYIH (SEQ ID NO: 31);
      (ii) a CDR2 comprising amino acid sequence RIDPENDETKYGPIFQG (SEQ ID NO: 32); and
      (iii) a CDR3 comprising amino acid sequence RGGVY (SEQ ID NO: 33); and
   (b) a light chain immunoglobulin variable region comprising:
      (i) a CDR1 comprising amino acid sequence KSSQSLLDSDGKTYLN (SEQ ID NO: 34);
      (ii) a CDR2 comprising the sequence LVSKLDS (SEQ ID NO: 35); and
      (iii) a CDR3 comprising amino acid sequence WQGTHFPGT (SEQ ID NO: 36).

In an embodiment of the dual targeting CAR-T cell, the humanized anti-EGFRvIII binding domain is a humanized single chain variable fragment (scFv) of monoclonal antibody clone 3C10, the humanized scFV having amino acid sequence:

(SEQ ID NO: 37)
EIQLVQSGAEVKKPGESLRISCKGSGFNIEDYYIHWVRQMPGKGLEWMG
RIDPENDETKYGPIFQGHVTISADTSINTVYLQWSSLKASDTAMYYCAF
RGGVYWGQGTTVTVSSGGGGSGGGGSGGGGSGGGGSDVVMTQSPDSLAV
SLGERATINCKSSQSLLDSDGKTYLNWLQQKPGQPPKRLISLVSKLDSG
VPDRFSGSGSGTDFTLTISSLQAEDVAVYYCWWQGTHFPGTFGGGTKVE
IK

In another embodiment, the second CAR further comprises:
(c) a transmembrane domain comprising amino acid sequence

(SEQ ID NO: 38)
IYIWAPLAGTCGVLLLSLVITLYC;

(d) a hinge region comprising amino acid sequence

(SEQ ID NO: 39)
TTTPAPRPPTPAPTIASQPLSLRPEACRPAAGGAVHTRGLDFACD;

(e) an intracellular signaling domain comprising a costimulatory domain and a primary signaling domain, wherein the costimulatory domain comprises a 4-1BB costimulatory domain comprising amino acid sequence of SEQ ID NO: 9.

In another embodiment of the dual targeting CAR-T cell, the primary signaling domain comprises a CD3 zeta domain having amino acid sequence:

(SEQ ID NO: 40)
RVKFSRSADAPAYKQGQNQLYNELNLGRREEYDVLDKRRGRDPEMGGKP
RRKNPQE.

In another embodiment, the primary signaling domain comprises amino acid sequence NCBI Reference Sequence NM_000734.3:

(SEQ ID NO: 41)
RVKFSRSADAPAYKQGQNQLYNELNLGRREEYDVLDKRRGRDPEMGGKP
RRKNPQELYNELQKDKMAEAYSEIGMKGERRRGKGHDGLYQGLSTATKD
TYDALHMQALPPR.

In another embodiment, a pharmaceutical composition comprises the dual targeting CAR-T cell that binds to human EphA3 and to human mutant EGFRvIII provided herein and a pharmaceutically acceptable carrier.

In another aspect, the present invention provides a method for producing a dual targeting CAR-T cell that binds to human EphA3 and human EGFRvIII, the method comprising: (a) transducing a T-cell with an EphA3-targeting CAR expression cassette comprising a CAR that binds to human EphA3 and is encoded by an isolated nucleic acid sequence of SEQ ID NO: 16, SEQ ID NO: 17, SEQ ID NO: 18 or SEQ ID NO: 19 to produce a human EphA3-targeting CAR T-cell; and (b) transducing the human EphA3-targeting CAR T-cell produced in step (a) with an expression cassette comprising a human EGFRvIII-targeting CAR that binds to human EGFRvIII and the human EGFRvIII-targeting CAR is encoded by a nucleic acid sequence of SEQ ID NO: 42:

ATGGCCTTACCAGTTACCGCCTTATTATTGCCTTTAGCCTTATTGTTAC
ATGCCGCCCGTCCGGGATCCGAGATTCAGCTGCAGCAATCTGGGGCAGA
ACTTGTGAAGCCAGGGGCCTCAGTCAAGCTGTCCTGCACAGGTTCTGGC
TTCAACATTGAAGACTACTATATTCACTGGGTGAAGCAGAGGACTGAAC
AGGGCCTGGAATGGATTGGAAGGATTGATCCTGAGAATGATGAAACTAA
ATATGGCCCAATATTCCAGGGCAGGGCCACTATAACAGCAGACACATCC
TCCAACACAGTCTACCTGCAACTCAGCAGCCTGACATCTGAGGACACTG
CCGTCTATTACTGTGCCTTTCGCGGTGGAGTCTACTGGGGGCCAGGAAC
CACTCTCACAGTCTCCTCAGGAGGTGGTGGTTCCGGTGGTGGTGGTTCC
GGAGGTGGTGGTTCACATATGGATGTTGTGATGACCCAGTCTCCACTCA
CTCTATCGGTTGCCATTGGACAATCAGCCTCCATCTCTTGCAAGTCAAG
TCAGAGCCTCTTAGATAGTGATGGAAAGACATATTTGAATTGGTTGTTA
CAGAGGCCAGGCCAGTCTCCAAAGCGCCTAATCTCTCTGGTGTCTAAAC
TGGACTCTGGAGTCCCTGACAGGTTCACTGGCAGTGGATCAGGGACAGA
TTTCACACTGAGAATCAGCAGAGTGGAGGCTGAGGATTTGGGAATTTAT
TATTGCTGGCAAGGTACACATTTTCCTGGGACGTTCGGTGGAGGGACCA
AGCTGGAGATAAAAGCTAGCACCACTACCCCTGCACCGCGACCACCAAC
ACCGGCGCCCACCATTGCGTCGCAGCCTCTGTCCCTGCGCCCAGAAGCA
TGCCGTCCAGCAGCAGGTGGTGCAGTTCATACTCGTGGTCTGGATTTCG
CCTGTGATATCTACATCTGGGCGCCCTTGGCCGGGACTTGTGGGGTCCT
TCTCCTGTCACTGGTTATCACCCTTTACTGCAAACGGGGCAGAAAGAAA
CTCCTGTATATATTCAAACAACCATTTATGAGACCAGTACAAACTACTC
AAGAGGAAGATGGCTGTAGCTGCCGATTTCCAGAAGAAGAAGAAGGAGG
ATGTGAACTGAGAGTGAAGTTCAGCAGGAGCGCAGACGCCCCCGCGTAC
AAGCAGGGCCAGAACCAGCTCTATAACGAGCTCAATCTAGGACGAAGAG
AGGAGTACGATGTTTTGGACAAGAGACGTGGCCGGGACCCTGAGATGGG
GGGAAAGCCGAGAAGGAAGAACCCTCAGGAAGGCCTGTACAATGAACTG
CAGAAAGATAAGATGGCGGAGGCCTACAGTGAGATTGGGATGAAAGGCG
AGCGCCGGAGGGGCAAGGGGCACGATGGCCTTTACCAGGGTCTCAGTAC
AGCCACCAAGGACACCTACGACGCCCTTCACATGCAGGCCCTGCCCCCT
CGCTAA

to produce a human EphA3-targeting- and a human EGFRvIII-targeting-CAR T-cell, wherein the human EphA3-targeting- and human EGFRvIII-targeting-CAR T-cell expresses the human EphA3-targeting CAR and the human EGFRvIII-targeting CAR on a cell surface thereof.

In an embodiment of the method, the encoded human EGFRvIII-targeting CAR comprises an amino acid sequence of SEQ ID NO: 43:

MALPVTALLLPLALLLHAARPGSEIQLQQSGAELVKPGASVKLSCTGSG
FNIEDYYIHWVKQRTEQGLEWIGRIDPENDETKYGPIFQGRATITADTS
SNTVYLQLSSLTSEDTAVYYCAFRGGVYWGPGTTLTVSSGGGGSGGGGS
GGGGSHMDVVMTQSPLTLSVAIGQSASISCKSSQSLLDSDGKTYLNWLL
QRPGQSPKRLISLVSKLDSGVPDRFTGSGSGTDFTLRISRVEAEDLGIY
YCWQGTHFPGTFGGGTKLEIKASTTTPAPRPPTPAPTIASQPLSLRPEA
CRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPP
R.

In another embodiment, provided is a method for treating a solid tumor cancer, the method comprising administering to a subject in need thereof the pharmaceutical composition, as described herein.

In a particular embodiment, the solid tumor cancer is a brain cancer. In a specific embodiment, the brain cancer is glioblastoma multiforme (GBM). In an embodiment, the solid tumor cancer is a colon, kidney, bladder, breast, liver, pancreatic, prostate tumor, a melanoma, myeloma or lung cancer, e.g., a non-small cell lung cancer (NSCLC) or a small cell lung cancer (SCLC). In another embodiment, the solid tumor cancer expresses EphA3 on a surface of tumor cells, tumor-associated vasculature and/or tumor stroma. In an embodiment, tumor stroma cells comprise myeloid derived suppressor cells. In an embodiment, the tumor cells comprise tumor stem cells. In particular embodiments, the tumor-associated vasculature comprises neo-vasculature.

In an embodiment, the method further comprises administering a human GM-CSF (hGM-CSF) antagonist selected from the group consisting of an anti-human GM-CSF antibody, an anti-hGM-CSF antibody fragment, a soluble hGM-CSF receptor alpha, an anti-hGM-CSF receptor (GM-CSFr) antibody and an anti-hGM-CSFr antibody fragment. In a specific embodiment, the anti-human GM-CSF antibody is hGM-CSF neutralizing antibody lenzilumab. In a specific embodiment the anti-human GM-CSF antibody is Namilumab, Otilimab, Gimsilumab, and TJM2 (TJ003234). In a further embodiment, the GM-CSF antagonist is anti-GM-CSF receptor antibody Mavrilimumab. In an embodiment, administration of lenzilumab reduces relapse rate or prevents occurrence of solid tumor relapse. In another embodiment, administration of lenzilumab prevents or reduces incidence of immunotherapy-related toxicity in the subject. In some embodiments, the immunotherapy-related toxicity is EphA3-targeting CAR-T cell related toxicity. In an embodiment, the EphA3-targeting CAR-T cell related toxicity is cytokine release syndrome, neurotoxicity and/or neuro-inflammation. In another embodiment, the method further comprises administering GM-CSF silenced EphA3-targeting CAR-T cells or gene knockout (GM-CSF^{k/o}) EphA3-targeting CAR-T cells, wherein administration of GM-CSF silenced EphA3-targeting CAR-T cells or GM-CSF^{k/o} EphA3-targeting CAR-T cells prevents or reduces incidence of immunotherapy-related toxicity in the subject. In an embodiment, administration of the GM-CSF silenced EphA3-targeting CAR-T cells or GM-CSF^{k/o} EphA3-targeting CAR-T cells reduces relapse rate or prevents occurrence of solid tumor relapse. In another embodiment, the method further comprises administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody. In an embodiment, the method further comprises administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody. In a specific embodiment the anti-programmed cell death-1 antibody is Pembrolizumab. In another embodiment the anti-programmed death-ligand antibody is avelumab. In another embodiment the anti-cytotoxic T-lymphocyte associated protein 4 (CTLA-4) antibody is Ipilimumab

In another embodiment, the immunotherapy-related toxicity is EphA3-targeting CAR-T cell related toxicity and/or human EGFRvIII targeting CAR-T cell related toxicity. In an embodiment, the EphA3-targeting CAR-T cell related toxicity and/or human EGFRvIII targeting CAR-T cell related toxicity is cytokine release syndrome, neurotoxicity and/or neuro-inflammation.

In an embodiment, the CAR that binds to human Eph receptor A3 (EphA3) comprises amino acid sequence:

(SEQ ID NO: 12)
MALPVTALLLPLALLLHAARPQVQLVQSGAEVKKPGASVKVSCKASGYT
FTGYWMNWVRQAPGQGLEWMGDIYPGSGNTNYDEKFQGRVTMTRDTSIS
TAYMELSRLRSDDTAVYYCARGGYYEDFDSWGQGTTVTVSSGGGGSGGG
GSGGGGSDIQMTQSPSFLSASVGDRVTITCRASQGIISYLAWYQQKPEK
APKRLIYAASSLQSGVPSRFSGSGSGTEFTLTISSLQPEDFATYYCGQY
ANYPYTFGQGTKLEIKTTTPAPRPPTPAPTIASQPLSLRPEACRPAAGG
AVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR.

In another embodiment, the CAR that binds to human Eph receptor A3 (EphA3) comprises amino acid sequence:

(SEQ ID NO: 13)
MALPVTALLLPLALLLHAARPQVQLVQSGAEVKKPGASVKVSCKASGYT
FTGYWMNWVRQAPGQGLEWMGDIYPGSGNTNYDEKFQGRVTMTRDTSIS
TAYMELSRLRSDDTAVYYCARGGYYEDFDSWGQGTTVTVSSGGGGSGGG
GSGGGGSDIQMTQSPSFLSASVGDRVTITCRASQGIISYLAWYQQKPEK
APKRLIYAASSLQSGVPSRFSGSGSGTEFTLTISSLQPEDFATYYCGQY
ANYPYTFGQGTKLEIKLEPKSCDKTHTCPPCPDPKFWVLVVVGGVLACY
SLLVTVAFIIFWVRSKRSRLLHSDYMNMTPRRPGPTRKHYQPYAPPRDF
AAYRSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPR.

In an embodiment, the CAR that binds to human Eph receptor A3 (EphA3) comprises amino acid sequence:

(SEQ ID NO: 14)
MALPVTALLLPLALLLHAARPDIQMTQSPSFLSASVGDRVTITCRASQG
IISYLAWYQQKPEKAPKRLIYAASSLQSGVPSRFSGSGSGTEFTLTISS
LQPEDFATYYCGQYANYPYTFGQGTKLEIKGGGGSGGGGSGGGGSQVQL
VQSGAEVKKPGASVKVSCKASGYTFTGYWMNWVRQAPGQGLEWMGDIYP
GSGNTNYDEKFQGRVTMTRDTSISTAYMELSRLRSDDTAVYYCARGGYY
EDFDSWGQGTTVTVSSLEPKSCDKTHTCPPCPDPKFWVLVVVGGVLACY
SLLVTVAFIIFWVRSKRSRLLHSDYMNMTPRRPGPTRKHYQPYAPPRDF
AAYRSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPR.

In an embodiment, the CAR that binds to human Eph receptor A3 (EphA3) comprises amino acid sequence:

(SEQ ID NO: 15)
MALPVTALLLPLALLLHAARPDIQMTQSPSFLSASVGDRVTITCRASQG
IISYLAWYQQKPEKAPKRLIYAASSLQSGVPSRFSGSGSGTEFTLTISS
LQPEDFATYYCGQYANYPYTFGQGTKLEIKGGGGSGGGGSGGGGSQVQL
VQSGAEVKKPGASVKVSCKASGYTFTGYWMNWVRQAPGQGLEWMGDIYP
GSGNTNYDEKFQGRVTMTRDTSISTAYMELSRLRSDDTAVYYCARGGYY
EDFDSWGQGTTVTVSSTTTPAPRPPTPAPTIASQPLSLRPEACRPAAGG
AVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR.

In another aspect, the present invention provides a chimeric antigen receptor (CAR) construct that binds to human Eph receptor A3 (EphA3), the CAR comprising an extracellular anti-human EphA3 binding domain comprising a human EphA3 single chain variable fragment (scFv) of anti-EphA3 monoclonal antibody, wherein the human EphA3 scFv comprises CDR3 of the V_{H} region comprises GGYYEDFDS (SEQ ID NO: 44) and the CDR3 of the V_{L} region comprises GQYANYPYT (SEQ ID NO:45).

In one aspect, the present invention provides a chimeric antigen receptor (CAR) construct that binds to human Eph receptor A3 (EphA3), the CAR construct comprising an extracellular anti-human EphA3 binding domain comprising:
a V_{H} region comprising amino acid sequence
QVQLVQSGAEVKKPGASVKVSCKASGYTFTGYWMNWVRQAPGQGLEWMGD IYPGSGNTNYDEKFQGRVTMTRDTSISTAYMELSRLRSDDTAVYYCARGG YYEDFDSWGQGTTVTVSS (SEQ ID NO:46) and
a V_{L} region comprising amino acid sequence
DIQMTQSPSFLSASVGDRVTITCRASQGIISYLAWYQQKPEKAPKRLIYA ASSLQSGVPSRFSGSGSGTEFTLTISSLQPEDFATYYCGQYANYPYTFGQ GTKLEIK (SEQ ID NO:47).

In another aspect, the present invention provides a chimeric antigen receptor (CAR) construct that binds to human Eph receptor A3 (EphA3), the CAR construct comprising an extracellular anti-human EphA3 binding domain comprising:
a V_{H} region CDR1 having a sequence SYWIN (SEQ ID NO:48), a V_{H} region CDR2 having a sequence DIYPGSGNTNYDEKFKR (SEQ ID NO:49), a V_{H} region CDR3 having a sequence SGYYEDFDS (SEQ ID NO:50), and
a V_{L} region CDR1 having a sequence RASQEISGYLG (SEQ ID NO:51), a V_{L} region CDR2 having a sequence AASTLDS (SEQ ID NO:52), and a V_{L} region CDR3 having a sequence VQYANYPYT (SEQ ID NO:53).

In an embodiment of the herein provided human EphA3-targeting CAR T-cells comprising the above-described EphA3-targeting CAR constructs, the CAR-T cell expresses the CAR on a cell surface thereof. In another embodiment of the human EphA3-targeting CAR T-cell, the human EphA3-targeting CAR T-cell is an autologous EphA3-targeting CAR T-cell. In a further embodiment a pharmaceutical composition comprises the herein provided human EphA3-targeting CAR-T cells and a pharmaceutically acceptable carrier.

In an embodiment, provided herein is a method for treating a solid tumor cancer, the method comprising administering to a subject in need thereof any of the pharmaceutical compositions described herein.

### Pharmaceutical Compositions

Described herein are pharmaceutical compositions comprising compounds or therapeutic agent of the invention and one or more pharmaceutically acceptable carriers and methods of administering them. “Pharmaceutically acceptable carriers” include any excipient which is nontoxic to the cell or mammal being exposed thereto at the dosages and concentrations employed. The pharmaceutical composition may include one or more therapeutic agents. In an embodiment, the pharmaceutical composition comprises a therapeutic agent, which is an EphA3 CART cell comprising an EphA-3 CART construct described herein in Examples 1, 2A, 2B and 3-5. In specific embodiments, the therapeutic agent comprises EphA3 CART cell comprising any one of EphA-3 CART constructs EphA3 CAR K082 (H2L-BBz), EphA3 CAR K083 (H2L-28z), EphA3 CAR K084 (L2H-28z), or EphA3 CAR K085 (L2H BBz) and combinations thereof. In an embodiment, the EphA3 CART cells comprise an EphA-3 CART construct comprising EphA3 scFv heavy chain comprising the nucleic acid sequence of SEQ ID NO: 21 and the nucleic acid sequence of SEQ ID NO: 23.

In a particular embodiment, the pharmaceutical composition comprises a therapeutic agent, wherein the therapeutic agent comprises EphA3 CART cells comprising an EphA-3 CART construct, the EphA-3 CART construct comprising EphA3 scFv heavy chain amino acid sequence
QVQLVQSGAEVKKPGASVKVSCKASGYTFTGYWMNWVRQAPGQGLEWM GDIYPGSGNTNYDEKFQGRVTMTRDTSISTAYMELSRLRSDDTAVYYCARG GYYEDFDSWGQGTTVTVSS (SEQ ID NO: 1) and EphA3 scFv light chain amino acid sequence
DIQMTQSPSFLSASVGDRVTITCRASQGIISYLAWYQQKPEK APKRLIYAASSLQSGVPSRFSGSGSGTEFTLTISSLQPEDFATY YCGQYANYPYTFGQGTKLEIK (SEQ ID NO: 2).

In a particular embodiment, the pharmaceutical composition comprises a therapeutic agent, which is an EphA-3 CART construct comprising the nucleic acid sequence of SEQ ID NO: 55, wherein the nucleic acid sequence comprises a CD8 leader, a single chain variable fragment (scFv) of anti-human EphA3 monoclonal antibody ifabotuzumab (Ifab scFv) and a CD8 hinge.

In some embodiments, the pharmaceutical composition comprises as the therapeutic agent EphA3 CART cells comprising a chimeric antigen receptor (CAR) construct that binds to human Eph receptor A3 (EphA3) using a human EphA3 single chain variable fragment (scFv) of anti-EphA3 monoclonal antibody, wherein the human EphA3 scFv comprises CDR3 of the V_{H} region comprises GGYYEDFDS (SEQ ID NO:44) and the CDR3 of the V_{L} region comprises GQYANYPYT (SEQ ID NO:45).

In certain embodiments, the pharmaceutical composition comprises as the therapeutic agent EphA3 CART cells comprising a chimeric antigen receptor (CAR) construct that binds to human Eph receptor A3 (EphA3), wherein the CAR construct comprises an extracellular anti-human EphA3 binding domain comprising:
a V_{H} region comprising amino acid sequence:
   QVQLVQSGAEVKKPGASVKVSCKASGYTFTGYWMNWVRQAPGQGLEWMGDIY PGSGNTNYDEKFQGRVTMTRDTSISTAYMELSRLRSDDTAVYYCARGGYYEDFD SWGQGTTVTVSS (SEQ ID NO:46) and
a V_{L} region comprising amino acid sequence:
   DIQMTQSPSFLSASVGDRVTITCRASQGIISYLAWYQQKPEKAPKRLIYA ASSLQSGVPSRFSGSGSGTEFTLTISSLQPEDFATYYCGQYANYPYTFGQ

GTKLEIK (SEQ ID NO:47). In an embodiment, the pharmaceutical composition comprises as the therapeutic agent EphA3 CART cells, the EphA3 CART cells comprising a chimeric antigen receptor (CAR) construct that binds to human Eph receptor A3 (EphA3), an extracellular anti-human EphA3 binding domain comprising a V_{H} region CDR1 having a sequence SYWIN (SEQ ID NO:48), a V_{H} region CDR2 having a sequence DIYPGSGNTNYDEKFKR (SEQ ID NO:49), a V_{H} region CDR3 having a sequence SGYYEDFDS (SEQ ID NO:50), and a V_{L} region CDR1 having a sequence RASQEISGYLG (SEQ ID NO:51), a V_{L} region CDR2 having a sequence AASTLDS (SEQ ID NO:52), and a V_{L} region CDR3 having a sequence VQYANYPYT (SEQ ID NO:53).

In an embodiment, the therapeutic agent comprises an EGFRViii CART (3C10-41BBz) comprising the nucleic acid sequence of SEQ ID NO: 42.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, or intra-vaginally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL™ (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The compositions and formulations as described herein may be administered alone or with other biologically active agents. Administration can be systemic or local, e.g. through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

### Effective Doses

Effective doses of the pharmaceutical compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule or therapeutic agent may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including retinal inflammation and neuron death secondary to ocular hypertension, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the dosage of the EphA3 CART cells comprising an EphA-3 CART construct as described herein—or combinations thereof, will be within the range of about 0.01 to-about 1000 mg/kg of body weight. In another embodiment, the dosage will be within the range of about 0.1 mg/kg to about 100 mg/kg. In another embodiment, the dosage will be within the range of about 1 mg/kg to about 10 mg/kg. In an embodiment, the dosage is about 10 mg/kg. In another embodiment, the dosage is 10 mg/kg.

The compound or composition or therapeutic agent of the invention, including the EphA3 CART cells comprising an EphA-3 CART construct as described herein—or combinations thereof, may be administered only once, or it may be administered multiple times. For multiple dosages, the composition may be, for example, administered three times a day, twice a day, once a day, once every two days, twice a week, weekly, once every two weeks, or monthly.

In an embodiment, the dosage is administered once. In an embodiment, the CAR-T cells according to the invention are administered once after lymphodepleting chemotherapy with Cyclophosphamide (CY) and Fludarabine (FLU). In an embodiment, the dosage is between 1×10⁴ cell/kg to 2×10⁶ cells/kg and is once. Moreover, effective doses may be extrapolated from dose-response curves derived from in vitro or animal model test bioassays or systems.

In an embodiment, the pharmaceutical composition comprising the human EphA3-targeting CAR-T cell is administered in combination with two or more pharmaceutical compositions, wherein each pharmaceutical composition comprises an antigen targeting CAR T cell, the antigen targeting CAR T cell comprising an antigen targeting CAR, wherein the antigen targeted by the CAR is selected from the group consisting of CAIX, CD133, CEA, c-MET, EGFR, EGFRvIII, EpCam, EphA2, ErB2/Her2, FAP, folate receptor alpha (FR-a), GD2, GPC3, IL-13Ra2, L1-CAM, Mesothelin, MUC1, PD-L1, PSCA, PSMA, ROR1, VEGFR-2, and HER2, or wherein each pharmaceutical composition comprises a liquid cancer CART selected from the group consisting of CART19, CART22, CART123, CART33, CART-CLL1, CART BCMA, CART38, CART138, and CS1-CART.

In an embodiment, the pharmaceutical composition comprising the human EphA3-targeting CAR-T cell is administered in combination with two or more pharmaceutical compositions, wherein each pharmaceutical composition comprises an antigen targeting CAR T cell, the antigen targeting CAR T cell comprising an antigen targeting CAR, wherein the antigen targeted is selected from the group consisting of CAIX, CD133, CEA, c-MET, EGFR, EGFRvIII, EpCam, EphA2, ErB2/Her2, FAP, folate receptor alpha (FR-a), GD2, GPC3, IL-13Ra2, L1-CAM, Mesothelin, MUC1, PD-L1, PSCA, PSMA, ROR1, VEGFR-2, and HER2, or wherein each pharmaceutical composition comprises a liquid cancer CART selected from the group consisting of CART19, CART22, CART123, CART33, CART-CLL1, CART BCMA, CART38, CART138, and CS1-CART.

In still another embodiment, the pharmaceutical composition comprising the human EphA3-targeting CAR-T cell is administered in combination with two or more pharmaceutical compositions, wherein each pharmaceutical composition comprises an antigen targeting CAR T cell, the antigen targeting CAR T cell comprising an antigen targeting CAR, wherein the antigen targeted is selected from the group consisting of CAIX, CD133, CEA, c-MET, EGFR, EGFRvIII, EpCam, EphA2, ErB2/Her2, FAP, folate receptor alpha (FR-a), GD2, GPC3, IL-13Ra2, L1-CAM, Mesothelin, MUC1, PD-L1, PSCA, PSMA, ROR1, VEGFR-2, and HER2, or wherein each pharmaceutical composition comprises a liquid cancer CART selected from the group consisting of CART19, CART22, CART123, CART33, CART-CLL1, CART BCMA, CART38, CART138, and CS1-CART. In various embodiments of the herein provided methods, the pharmaceutical composition comprising the human EphA3-targeting CAR-T cell is administered together or sequentially with the two or more pharmaceutical compositions. In an embodiment, administration of the pharmaceutical composition comprising the human EphA3-targeting CAR-T cell and the two or more pharmaceutical compositions improves objective response rates of the subject. In another embodiment, administration of the pharmaceutical composition comprising the human EphA3-targeting CAR-T cell and the two or more pharmaceutical compositions improves progression free survival of the subject. In yet another embodiment, administration of the pharmaceutical composition comprising the human EphA3-targeting CAR-T cell and the two or more pharmaceutical compositions improves overall survival of the subject. In an embodiment of the dual targeting CAR-T cell that binds to human EphA3 and to human mutant EGFRvIII as described herein, the dual targeting CAR-T cell is an autologous EphA3-targeting CAR T-cell. In an embodiment the herein provided pharmaceutical compositions comprise the dual targeting CAR-T cells that binds to human EphA3 and to human mutant EGFRvIII, as provided and described herein and a pharmaceutically acceptable carrier. In another embodiment of the herein provided and described methods for treating a solid tumor cancer, the method comprises administering to a subject in need thereof any one of the pharmaceutical compositions provided herein.

All patents and literature references cited in the present specification are hereby incorporated by reference in their entirety.

The following examples are presented in order to illustrate certain embodiments of the invention more fully. The examples should in no way be construed, however, as limiting the broad scope of the invention.

### EXAMPLES

### Example 1

### Construction of CARs Binding to EphA3 and to Binding to EGFRViii

We constructed four chimeric antigen receptors (CARs) targeting EphA3 using the following nucleic acid sequences of a leader sequence, EphA3 scFv heavy chain, a linker sequence, EphA3 scFv light chain, hinge regions, transmembrane (TM) domains, intracellular signaling domains comprising a 4-1BB costimulatory domain or a CD28 costimulatory domain and aCD3 zeta activation domain. We constructed a CAR targeting EGFRViii as described in U.S. Pat. No. 9,394,368, which is incorporated herein in its entirety. The following nucleic acid sequence (SEQ ID NO: 42) was used, which encoded the EGFRViii of SEQ ID NO: 43.

CD8 leader:
(SEQ ID NO: 20)
ATGGCCTTACCAGTGACCGCCTTGCTCCTGCCGCTGGCCTTGCTGCTCCA
CGCCGCCAGGCCG
EphA3 scFv heavy chain:
(SEO ID NO: 21)
CAGGTGCAGCTGGTGCAGTCTGGGGCTGAGGTGAAGAAGCCTGGGGCCTC
AGTGAAGGTTTCCTGCAAGGCTTCTGGATACACCTTCACAGGCTACTGGA
TGAATTGGGTGCGCCAGGCCCCCGGGCAAGGCCTGGAGTGGATGGGGGAC
ATCTACCCGGGCAGTGGTAACACAAACTACGATGAGAAGTTCCAGGGTAG
AGTCACGATGACCAGGGACACGTCCATCAGCACAGCCTACATGGAGCTGA
GCAGGCTGAGATCTGACGACACAGCCGTGTACTACTGCGCAAGAGGTGGA
TATTATGAAGATTTTGATAGCTGGGGCCAAGGTACCACTGTGACCGTGAG
CTCC
Linker
(SEQ ID NO: 22)
GGTGGAGGTGGTTCGGGAGGTGGAGGTAGCGGAGGTGGTGGATCT
EphA3 scFv light chain
(SEQ ID NO: 23)
GACATCCAGATGACCCAGTCTCCATCCTTCCTGTCTGCATCTGTAGGAGA
CAGAGTCACCATCACTTGCCGGGCCAGTCAGGGCATTATCAGTTATTTAG
CCTGGTATCAGCAAAAACCAGAGAAAGCCCCTAAGCGCCTGATCTATGCT
GCATCCAGTTTGCAAAGTGGGGTCCCATCAAGGTTCAGCGGCAGTGGATC
TGGGACAGAATTCACTCTCACAATCAGCAGCCTGCAGCCTGAAGATTTTG
CAACTTACTACTGCGGGCAGTATGCCAATTATCCGTACACCTTTGGCCAA
GGTACGAAACTGGAAATTAAA
CD8 hinge
SEQ ID NO: 24)
ACCACTACCCCTGCACCGCGACCACCAACACCGGCGCCCACCATTGCGTC
GCAGCCTCTGTCCCTGCGCCCAGAAGCATGCCGTCCAGCAGCAGGTGGTG
CAGTTCATACTCGTGGTCTGGATTTCGCCTGTGAT
CD8 TM
SEQ ID NO: 25)
ATCTACATCTGGGCGCCCTTGGCCGGGACTTGTGGGGTCCTTCTCCTGTC
ACTGGTTATCACCCTTTACTGC
41BB
(SEQ ID NO: 26)
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
CD28 hinge
SEQ ID NO: 27)
CTCGAGCCCAAATCTTGTGACAAAACTCACACATGCCCACCGTGCCCGGA
TCCCAAA
CD28 TM
(SEQ ID NO: 28)
TTTTGGGTGCTGGTGGTGGTTGGTGGAGTCCTGGCTTGCTATAGCTTGCT
AGTAACAGTGGCCTTTATTATTTTCTGGGTG
CD28
(SEQ ID NO: 29)
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC
CD3z
(SEQ ID NO: 30)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

The above-provided isolated nucleic acid sequences encoded the following amino acid sequences for the herein described CARs:

CD8 leader amino acid sequence
(SEQ ID NO: 3)
MALPVTALLLPLALLLHAARP
EphA3 scFv heavy chain amino acid sequence
(SEQ ID NO: 1)
QVQLVQSGAEVKKPGASVKVSCKASGYTFTGYWMNWVRQAPGQGLEWMGD
IYPGSGNTNYDEKFQGRVTMTRDTSISTAYMELSRLRSDDTAVYYCARGG
YYEDFDSWGQGTTVTVSS
Linker amino acid sequence
(SEQ ID NO: 4)
GGGGSGGGGSGGGGS
EphA3 scFv light chain amino acid sequence
(SEQ ID NO: 2)
DIQMTQSPSFLSASVGDRVTITCRASQGIISYLAWYQQKPEKAPKRLIYA
ASSLQSGVPSRFSGSGSGTEFTLTISSLQPEDFATYYCGQYANYPYTFGQ
GTKLEIK
CD8 hinge amino acid sequence
(SEQ ID NO: 5)
TTTPAPRPPTPAPTIASQPLSLRPEACRPAAGGAVHTRGLDFACD
CD8 TM amino acid sequence
(SEQ ID NO: 7)
IYIWAPLAGTCGVLLLSLVITLYC
41BB amino acid sequence
(SEQ ID NO: 9)
KRGRKKLLYIFKQPFMRPVQTTQEEDGCSCRFPEEEEGGCEL
CD28 hinge amino acid sequence
(SEQ ID NO: 6)
LEPKSCDKTHTCPPCPDPK
CD28 TM amino acid sequence
(SEQ ID NO: 8)
FWVLVVVGGVLACYSLLVTVAFIIFWV
CD28 amino acid sequence
(SEQ ID NO: 10)
RSKRSRLLHSDYMNMTPRRPGPTRKHYQPYAPPRDFAAYRS
CD3z amino acid sequence
(SEQ ID NO: 11)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The following isolated nucleic acid sequence encoded the herein described EphA3-targeting CARs:

EphA3 CAR K082 (H2L-BBz)
(SEQ ID NO: 16)
ATGGCCTTACCAGTGACCGCCTTGCTCCTGCCGCTGGCCTTGCTGCTCCACGCCG
CCAGGCCGCAGGTGCAGCTGGTGCAGTCTGGGGCTGAGGTGAAGAAGCCTGGGG
CCTCAGTGAAGGTTTCCTGCAAGGCTTCTGGATACACCTTCACAGGCTACTGGAT
GAATTGGGTGCGCCAGGCCCCCGGGCAAGGCCTGGAGTGGATGGGGGACATCTA
CCCGGGCAGTGGTAACACAAACTACGATGAGAAGTTCCAGGGTAGAGTCACGAT
GACCAGGGACACGTCCATCAGCACAGCCTACATGGAGCTGAGCAGGCTGAGATC
TGACGACACAGCCGTGTACTACTGCGCAAGAGGTGGATATTATGAAGATTTTGAT
AGCTGGGGCCAAGGTACCACTGTGACCGTGAGCTCCGGTGGAGGTGGTTCGGGA
GGTGGAGGTAGCGGAGGTGGTGGATCTGACATCCAGATGACCCAGTCTCCATCC
TTCCTGTCTGCATCTGTAGGAGACAGAGTCACCATCACTTGCCGGGCCAGTCAGG
GCATTATCAGTTATTTAGCCTGGTATCAGCAAAAACCAGAGAAAGCCCCTAAGC
GCCTGATCTATGCTGCATCCAGTTTGCAAAGTGGGGTCCCATCAAGGTTCAGCGG
CAGTGGATCTGGGACAGAATTCACTCTCACAATCAGCAGCCTGCAGCCTGAAGA
TTTTGCAACTTACTACTGCGGGCAGTATGCCAATTATCCGTACACCTTTGGCCAA
GGTACGAAACTGGAAATTAAAACCACTACCCCTGCACCGCGACCACCAACACCG
GCGCCCACCATTGCGTCGCAGCCTCTGTCCCTGCGCCCAGAAGCATGCCGTCCAG
CAGCAGGTGGTGCAGTTCATACTCGTGGTCTGGATTTCGCCTGTGATATCTACAT
CTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGAC
GCCCCCGCGTACAAGCAGGGCCAGAACCAGCTCTATAACGAGCTCAATCTAGGA
CGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGC
EphA3 CAR K083 (H2L-28z)
(SEQ ID NO: 17)
ATGGCCTTACCAGTGACCGCCTTGCTCCTGCCGCTGGCCTTGCTGCTCCACGCCG
CCAGGCCGCAGGTGCAGCTGGTGCAGTCTGGGGCTGAGGTGAAGAAGCCTGGGG
CCTCAGTGAAGGTTTCCTGCAAGGCTTCTGGATACACCTTCACAGGCTACTGGAT
GAATTGGGTGCGCCAGGCCCCCGGGCAAGGCCTGGAGTGGATGGGGGACATCTA
CCCGGGCAGTGGTAACACAAACTACGATGAGAAGTTCCAGGGTAGAGTCACGAT
GACCAGGGACACGTCCATCAGCACAGCCTACATGGAGCTGAGCAGGCTGAGATC
TGACGACACAGCCGTGTACTACTGCGCAAGAGGTGGATATTATGAAGATTTTGAT
AGCTGGGGCCAAGGTACCACTGTGACCGTGAGCTCCGGTGGAGGTGGTTCGGGA
GGTGGAGGTAGCGGAGGTGGTGGATCTGACATCCAGATGACCCAGTCTCCATCC
TTCCTGTCTGCATCTGTAGGAGACAGAGTCACCATCACTTGCCGGGCCAGTCAGG
GCATTATCAGTTATTTAGCCTGGTATCAGCAAAAACCAGAGAAAGCCCCTAAGC
GCCTGATCTATGCTGCATCCAGTTTGCAAAGTGGGGTCCCATCAAGGTTCAGCGG
CAGTGGATCTGGGACAGAATTCACTCTCACAATCAGCAGCCTGCAGCCTGAAGA
TTTTGCAACTTACTACTGCGGGCAGTATGCCAATTATCCGTACACCTTTGGCCAA
GGTACGAAACTGGAAATTAAACTCGAGCCCAAATCTTGTGACAAAACTCACACA
TGCCCACCGTGCCCGGATCCCAAATTTTGGGTGCTGGTGGTGGTTGGTGGAGTCC
TGGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACAAGCAGG
GCCAGAACCAGCTCTATAACGAGCTCAATCTAGGACGAAGAGAGGAGTACGATG
TTTTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGC
EphA3 CAR K084 (L2H-28z)
(SEQ ID NO: 18)
ATGGCCTTACCAGTGACCGCCTTGCTCCTGCCGCTGGCCTTGCTGCTCCACGCCG
CCAGGCCGGACATCCAGATGACCCAGTCTCCATCCTTCCTGTCTGCATCTGTAGG
AGACAGAGTCACCATCACTTGCCGGGCCAGTCAGGGCATTATCAGTTATTTAGCC
TGGTATCAGCAAAAACCAGAGAAAGCCCCTAAGCGCCTGATCTATGCTGCATCC
AGTTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTACTACTGCG
GGCAGTATGCCAATTATCCGTACACCTTTGGCCAAGGTACGAAACTGGAAATTA
AAGGTGGAGGTGGTTCGGGAGGTGGAGGTAGCGGAGGTGGTGGATCTCAGGTGC
AGCTGGTGCAGTCTGGGGCTGAGGTGAAGAAGCCTGGGGCCTCAGTGAAGGTTT
CCTGCAAGGCTTCTGGATACACCTTCACAGGCTACTGGATGAATTGGGTGCGCCA
GGCCCCCGGGCAAGGCCTGGAGTGGATGGGGGACATCTACCCGGGCAGTGGTAA
CACAAACTACGATGAGAAGTTCCAGGGTAGAGTCACGATGACCAGGGACACGTC
CATCAGCACAGCCTACATGGAGCTGAGCAGGCTGAGATCTGACGACACAGCCGT
GTACTACTGCGCAAGAGGTGGATATTATGAAGATTTTGATAGCTGGGGCCAAGG
TACCACTGTGACCGTGAGCTCCCTCGAGCCCAAATCTTGTGACAAAACTCACACA
TGCCCACCGTGCCCGGATCCCAAATTTTGGGTGCTGGTGGTGGTTGGTGGAGTCC
TGGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACAAGCAGG
GCCAGAACCAGCTCTATAACGAGCTCAATCTAGGACGAAGAGAGGAGTACGATG
TTTTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGC
EphA3 CAR K085 (L2H BBz)
(SEQ ID NO: 19)
ATGGCCTTACCAGTGACCGCCTTGCTCCTGCCGCTGGCCTTGCTGCTCCACGCCG
CCAGGCCGGACATCCAGATGACCCAGTCTCCATCCTTCCTGTCTGCATCTGTAGG
AGACAGAGTCACCATCACTTGCCGGGCCAGTCAGGGCATTATCAGTTATTTAGCC
TGGTATCAGCAAAAACCAGAGAAAGCCCCTAAGCGCCTGATCTATGCTGCATCC
AGTTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTACTACTGCG
GGCAGTATGCCAATTATCCGTACACCTTTGGCCAAGGTACGAAACTGGAAATTA
AAGGTGGAGGTGGTTCGGGAGGTGGAGGTAGCGGAGGTGGTGGATCTCAGGTGC
AGCTGGTGCAGTCTGGGGCTGAGGTGAAGAAGCCTGGGGCCTCAGTGAAGGTTT
CCTGCAAGGCTTCTGGATACACCTTCACAGGCTACTGGATGAATTGGGTGCGCCA
GGCCCCCGGGCAAGGCCTGGAGTGGATGGGGGACATCTACCCGGGCAGTGGTAA
CACAAACTACGATGAGAAGTTCCAGGGTAGAGTCACGATGACCAGGGACACGTC
CATCAGCACAGCCTACATGGAGCTGAGCAGGCTGAGATCTGACGACACAGCCGT
GTACTACTGCGCAAGAGGTGGATATTATGAAGATTTTGATAGCTGGGGCCAAGG
TACCACTGTGACCGTGAGCTCCACCACTACCCCTGCACCGCGACCACCAACACCG
GCGCCCACCATTGCGTCGCAGCCTCTGTCCCTGCGCCCAGAAGCATGCCGTCCAG
CAGCAGGTGGTGCAGTTCATACTCGTGGTCTGGATTTCGCCTGTGATATCTACAT
CTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGAC
GCCCCCGCGTACAAGCAGGGCCAGAACCAGCTCTATAACGAGCTCAATCTAGGA
CGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGC

The above-described nucleic acid sequences encode chimeric CARs comprising the following amino acid sequences:

EphA3 CAR K082 (H2L-BBz) amino acid sequence
(SEQ ID NO: 12)
MALPVTALLLPLALLLHAARPQVQLVQSGAEVKKPGASVKVSCKASGYTF
TGYWMNWVRQAPGQGLEWMGDIYPGSGNTNYDEKFQGRVTMTRDTSISTA
YMELSRLRSDDTAVYYCARGGYYEDFDSWGQGTTVTVSSGGGGSGGGGSG
GGGSDIQMTQSPSFLSASVGDRVTITCRASQGIISYLAWYQQKPEKAPKR
LIYAASSLQSGVPSRFSGSGSGTEFTLTISSLQPEDFATYYCGQYANYPY
TFGQGTKLEIKTTTPAPRPPTPAPTIASQPLSLRPEACRPAAGGAVHTRG
LDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR
EphA3 CAR K083 (H2L-28z) amino acid sequence
(SEQ ID NO: 13)
MALPVTALLLPLALLLHAARPQVQLVQSGAEVKKPGASVKVSCKASGYTF
TGYWMNWVRQAPGQGLEWMGDIYPGSGNTNYDEKFQGRVTMTRDTSISTA
YMELSRLRSDDTAVYYCARGGYYEDFDSWGQGTTVTVSSGGGGSGGGGSG
GGGSDIQMTQSPSFLSASVGDRVTITCRASQGIISYLAWYQQKPEKAPKR
LIYAASSLQSGVPSRFSGSGSGTEFTLTISSLQPEDFATYYCGQYANYPY
TFGQGTKLEIKLEPKSCDKTHTCPPCPDPKFWVLVVVGGVLACYSLLVTV
AFIIFWVRSKRSRLLHSDYMNMTPRRPGPTRKHYQPYAPPRDFAAYRSRV
KFSRSADAPAYKQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
EphA3 CAR K084 (L2H-28z) amino acid sequence
(SEQ ID NO: 14)
MALPVTALLLPLALLLHAARPDIQMTQSPSFLSASVGDRVTITCRASQGI
ISYLAWYQQKPEKAPKRLIYAASSLQSGVPSRFSGSGSGTEFTLTISSLQ
PEDFATYYCGQYANYPYTFGQGTKLEIKGGGGSGGGGSGGGGSQVQLVQS
GAEVKKPGASVKVSCKASGYTFTGYWMNWVRQAPGQGLEWMGDIYPGSGN
TNYDEKFQGRVTMTRDTSISTAYMELSRLRSDDTAVYYCARGGYYEDFDS
WGQGTTVTVSSLEPKSCDKTHTCPPCPDPKFWVLVVVGGVLACYSLLVTV
AFIIFWVRSKRSRLLHSDYMNMTPRRPGPTRKHYQPYAPPRDFAAYRSRV
KFSRSADAPAYKQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
EphA3 CAR K085 (L2H BBz) amino acid sequence
(SEQ ID NO: 15)
MALPVTALLLPLALLLHAARPDIQMTQSPSFLSASVGDRVTITCRASQGI
ISYLAWYQQKPEKAPKRLIYAASSLQSGVPSRFSGSGSGTEFTLTISSLQ
PEDFATYYCGQYANYPYTFGQGTKLEIKGGGGSGGGGSGGGGSQVQLVQS
GAEVKKPGASVKVSCKASGYTFTGYWMNWVRQAPGQGLEWMGDIYPGSGN
TNYDEKFQGRVTMTRDTSISTAYMELSRLRSDDTAVYYCARGGYYEDFDS
WGQGTTVTVSSTTTPAPRPPTPAPTIASQPLSLRPEACRPAAGGAVHTRG
LDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR

A CAR that binds to human EphA3 and to human mutant epidermal growth factor receptor variant III (EGFRvIII) was constructed.

We constructed a chimeric antigen receptor that binds to human mutant epidermal growth factor receptor variant III (EGFRvIII) using the following nucleic acid sequence, wherein the intracellular signaling domain comprises a 4-1BB costimulatory domain:

EGFRViii CART (3C10-41BBz)
(SEQ ID NO: 42)
ATGGCCTTACCAGTTACCGCCTTATTATTGCCTTTAGCCTTATTGTTACA
TGCCGCCCGTCCGGGATCCGAGATTCAGCTGCAGCAATCTGGGGCAGAAC
TTGTGAAGCCAGGGGCCTCAGTCAAGCTGTCCTGCACAGGTTCTGGCTTC
AACATTGAAGACTACTATATTCACTGGGTGAAGCAGAGGACTGAACAGGG
CCTGGAATGGATTGGAAGGATTGATCCTGAGAATGATGAAACTAAATATG
GCCCAATATTCCAGGGCAGGGCCACTATAACAGCAGACACATCCTCCAAC
ACAGTCTACCTGCAACTCAGCAGCCTGACATCTGAGGACACTGCCGTCTA
TTACTGTGCCTTTCGCGGTGGAGTCTACTGGGGGCCAGGAACCACTCTCA
CAGTCTCCTCAGGAGGTGGTGGTTCCGGTGGTGGTGGTTCCGGAGGTGGT
GGTTCACATATGGATGTTGTGATGACCCAGTCTCCACTCACTCTATCGGT
TGCCATTGGACAATCAGCCTCCATCTCTTGCAAGTCAAGTCAGAGCCTCT
TAGATAGTGATGGAAAGACATATTTGAATTGGTTGTTACAGAGGCCAGGC
CAGTCTCCAAAGCGCCTAATCTCTCTGGTGTCTAAACTGGACTCTGGAGT
CCCTGACAGGTTCACTGGCAGTGGATCAGGGACAGATTTCACACTGAGAA
TCAGCAGAGTGGAGGCTGAGGATTTGGGAATTTATTATTGCTGGCAAGGT
ACACATTTTCCTGGGACGTTCGGTGGAGGGACCAAGCTGGAGATAAAAGC
TAGCACCACTACCCCTGCACCGCGACCACCAACACCGGCGCCCACCATTG
CGTCGCAGCCTCTGTCCCTGCGCCCAGAAGCATGCCGTCCAGCAGCAGGT
GGTGCAGTTCATACTCGTGGTCTGGATTTCGCCTGTGATATCTACATCTG
GGCGCCCTTGGCCGGGACTTGTGGGGTCCTTCTCCTGTCACTGGTTATCA
CCCTTTACTGCAAACGGGGCAGAAAGAAACTCCTGTATATATTCAAACAA
CCATTTATGAGACCAGTACAAACTACTCAAGAGGAAGATGGCTGTAGCTG
CCGATTTCCAGAAGAAGAAGAAGGAGGATGTGAACTGAGAGTGAAGTTCA
GCAGGAGCGCAGACGCCCCCGCGTACAAGCAGGGCCAGAACCAGCTCTAT
AACGAGCTCAATCTAGGACGAAGAGAGGAGTACGATGTTTTGGACAAGAG
ACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGG
CCTTTACCAGGGTCTCAGTACAGCCACCAAGGACACCTACGACGCCCTTC
ACATGCAGGCCCTGCCCCCTCGCTAA.

The encoded chimeric antigen receptor (CAR) that binds to human mutant epidermal growth factor receptor variant III (EGFRvIII) comprises the following amino acid sequence:

(SEQ ID NO: 43)
MALPVTALLLPLALLLHAARPGSEIQLQQSGAELVKPGASVKLSCTGSGF
NIEDYYIHWVKQRTEQGLEWIGRIDPENDETKYGPIFQGRATITADTSSN
TVYLQLSSLTSEDTAVYYCAFRGGVYWGPGTTLTVSSGGGGSGGGGSGGG
GSHMDVVMTQSPLTLSVAIGQSASISCKSSQSLLDSDGKTYLNWLLQRPG
QSPKRLISLVSKLDSGVPDRFTGSGSGTDFTLRISRVEAEDLGIYYCWQG
THFPGTFGGGTKLEIKASTTTPAPRPPTPAPTIASQPLSLRPEACRPAAG
GAVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR.

### Examples

### Example 2A

### Methods

We developed a second generation CD28 co-stimulated CAR constructs in a third generation lentivirus backbone to generate EphA3 CART cells using the single chain variable fragment of ifabotuzumab, a monoclonal antibody directed against EphA3. Patient derived GBM xenograft cell lines were used in these experiments. The EphA3 CART construct was designed and then synthesized de novo using a commercially available protein synthesis vendor using the following DNA sequences, as were three additional EphA-3 CART constructs, respectively:

EphA3 CAR K082 (H2L-BBz)
(SEQ ID NO: 16)
ATGGCCTTACCAGTGACCGCCTTGCTCCTGCCGCTGGCCTTGCTGCTCCACGCCG
CCAGGCCGCAGGTGCAGCTGGTGCAGTCTGGGGCTGAGGTGAAGAAGCCTGGGG
CCTCAGTGAAGGTTTCCTGCAAGGCTTCTGGATACACCTTCACAGGCTACTGGAT
GAATTGGGTGCGCCAGGCCCCCGGGCAAGGCCTGGAGTGGATGGGGGACATCTA
CCCGGGCAGTGGTAACACAAACTACGATGAGAAGTTCCAGGGTAGAGTCACGAT
GACCAGGGACACGTCCATCAGCACAGCCTACATGGAGCTGAGCAGGCTGAGATC
TGACGACACAGCCGTGTACTACTGCGCAAGAGGTGGATATTATGAAGATTTTGAT
AGCTGGGGCCAAGGTACCACTGTGACCGTGAGCTCCGGTGGAGGTGGTTCGGGA
GGTGGAGGTAGCGGAGGTGGTGGATCTGACATCCAGATGACCCAGTCTCCATCC
TTCCTGTCTGCATCTGTAGGAGACAGAGTCACCATCACTTGCCGGGCCAGTCAGG
GCATTATCAGTTATTTAGCCTGGTATCAGCAAAAACCAGAGAAAGCCCCTAAGC
GCCTGATCTATGCTGCATCCAGTTTGCAAAGTGGGGTCCCATCAAGGTTCAGCGG
CAGTGGATCTGGGACAGAATTCACTCTCACAATCAGCAGCCTGCAGCCTGAAGA
TTTTGCAACTTACTACTGCGGGCAGTATGCCAATTATCCGTACACCTTTGGCCAA
GGTACGAAACTGGAAATTAAAACCACTACCCCTGCACCGCGACCACCAACACCG
GCGCCCACCATTGCGTCGCAGCCTCTGTCCCTGCGCCCAGAAGCATGCCGTCCAG
CAGCAGGTGGTGCAGTTCATACTCGTGGTCTGGATTTCGCCTGTGATATCTACAT
CTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGAC
GCCCCCGCGTACAAGCAGGGCCAGAACCAGCTCTATAACGAGCTCAATCTAGGA
CGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGC
EPHA3 CAR K083 (H2L-28z)
(SEQ ID NO: 17)
ATGGCCTTACCAGTGACCGCCTTGCTCCTGCCGCTGGCCTTGCTGCTCCACGCCG
CCAGGCCGCAGGTGCAGCTGGTGCAGTCTGGGGCTGAGGTGAAGAAGCCTGGGG
CCTCAGTGAAGGTTTCCTGCAAGGCTTCTGGATACACCTTCACAGGCTACTGGAT
GAATTGGGTGCGCCAGGCCCCCGGGCAAGGCCTGGAGTGGATGGGGGACATCTA
CCCGGGCAGTGGTAACACAAACTACGATGAGAAGTTCCAGGGTAGAGTCACGAT
GACCAGGGACACGTCCATCAGCACAGCCTACATGGAGCTGAGCAGGCTGAGATC
TGACGACACAGCCGTGTACTACTGCGCAAGAGGTGGATATTATGAAGATTTTGAT
AGCTGGGGCCAAGGTACCACTGTGACCGTGAGCTCCGGTGGAGGTGGTTCGGGA
GGTGGAGGTAGCGGAGGTGGTGGATCTGACATCCAGATGACCCAGTCTCCATCC
TTCCTGTCTGCATCTGTAGGAGACAGAGTCACCATCACTTGCCGGGCCAGTCAGG
GCATTATCAGTTATTTAGCCTGGTATCAGCAAAAACCAGAGAAAGCCCCTAAGC
GCCTGATCTATGCTGCATCCAGTTTGCAAAGTGGGGTCCCATCAAGGTTCAGCGG
CAGTGGATCTGGGACAGAATTCACTCTCACAATCAGCAGCCTGCAGCCTGAAGA
TTTTGCAACTTACTACTGCGGGCAGTATGCCAATTATCCGTACACCTTTGGCCAA
GGTACGAAACTGGAAATTAAACTCGAGCCCAAATCTTGTGACAAAACTCACACA
TGCCCACCGTGCCCGGATCCCAAATTTTGGGTGCTGGTGGTGGTTGGTGGAGTCC
TGGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACAAGCAGG
GCCAGAACCAGCTCTATAACGAGCTCAATCTAGGACGAAGAGAGGAGTACGATG
TTTTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGC
EPHA3 CAR K084 (L2H-28z)
(SEQ ID NO: 18)
ATGGCCTTACCAGTGACCGCCTTGCTCCTGCCGCTGGCCTTGCTGCTCCACGCCG
CCAGGCCGGACATCCAGATGACCCAGTCTCCATCCTTCCTGTCTGCATCTGTAGG
AGACAGAGTCACCATCACTTGCCGGGCCAGTCAGGGCATTATCAGTTATTTAGCC
TGGTATCAGCAAAAACCAGAGAAAGCCCCTAAGCGCCTGATCTATGCTGCATCC
AGTTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTACTACTGCG
GGCAGTATGCCAATTATCCGTACACCTTTGGCCAAGGTACGAAACTGGAAATTA
AAGGTGGAGGTGGTTCGGGAGGTGGAGGTAGCGGAGGTGGTGGATCTCAGGTGC
AGCTGGTGCAGTCTGGGGCTGAGGTGAAGAAGCCTGGGGCCTCAGTGAAGGTTT
CCTGCAAGGCTTCTGGATACACCTTCACAGGCTACTGGATGAATTGGGTGCGCCA
GGCCCCCGGGCAAGGCCTGGAGTGGATGGGGGACATCTACCCGGGCAGTGGTAA
CACAAACTACGATGAGAAGTTCCAGGGTAGAGTCACGATGACCAGGGACACGTC
CATCAGCACAGCCTACATGGAGCTGAGCAGGCTGAGATCTGACGACACAGCCGT
GTACTACTGCGCAAGAGGTGGATATTATGAAGATTTTGATAGCTGGGGCCAAGG
TACCACTGTGACCGTGAGCTCCCTCGAGCCCAAATCTTGTGACAAAACTCACACA
TGCCCACCGTGCCCGGATCCCAAATTTTGGGTGCTGGTGGTGGTTGGTGGAGTCC
TGGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACAAGCAGG
GCCAGAACCAGCTCTATAACGAGCTCAATCTAGGACGAAGAGAGGAGTACGATG
TTTTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGC
EPHA3 CAR K085 (L2H BBz)
(SEQ ID NO: 19)
ATGGCCTTACCAGTGACCGCCTTGCTCCTGCCGCTGGCCTTGCTGCTCCACGCCG
CCAGGCCGGACATCCAGATGACCCAGTCTCCATCCTTCCTGTCTGCATCTGTAGG
AGACAGAGTCACCATCACTTGCCGGGCCAGTCAGGGCATTATCAGTTATTTAGCC
TGGTATCAGCAAAAACCAGAGAAAGCCCCTAAGCGCCTGATCTATGCTGCATCC
AGTTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTACTACTGCG
GGCAGTATGCCAATTATCCGTACACCTTTGGCCAAGGTACGAAACTGGAAATTA
AAGGTGGAGGTGGTTCGGGAGGTGGAGGTAGCGGAGGTGGTGGATCTCAGGTGC
AGCTGGTGCAGTCTGGGGCTGAGGTGAAGAAGCCTGGGGCCTCAGTGAAGGTTT
CCTGCAAGGCTTCTGGATACACCTTCACAGGCTACTGGATGAATTGGGTGCGCCA
GGCCCCCGGGCAAGGCCTGGAGTGGATGGGGGACATCTACCCGGGCAGTGGTAA
CACAAACTACGATGAGAAGTTCCAGGGTAGAGTCACGATGACCAGGGACACGTC
CATCAGCACAGCCTACATGGAGCTGAGCAGGCTGAGATCTGACGACACAGCCGT
GTACTACTGCGCAAGAGGTGGATATTATGAAGATTTTGATAGCTGGGGCCAAGG
TACCACTGTGACCGTGAGCTCCACCACTACCCCTGCACCGCGACCACCAACACCG
GCGCCCACCATTGCGTCGCAGCCTCTGTCCCTGCGCCCAGAAGCATGCCGTCCAG
CAGCAGGTGGTGCAGTTCATACTCGTGGTCTGGATTTCGCCTGTGATATCTACAT
CTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGAC
GCCCCCGCGTACAAGCAGGGCCAGAACCAGCTCTATAACGAGCTCAATCTAGGA
CGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGC

The EphA3 CAR construct was subsequently cloned into a third generation lentivirus under control of an EF-1α promoter. The single chain variable region fragment is a proprietary sequence produced by Humanigen. The EphA3 construct possesses a second generation CD28 costimulatory domain and CD3ζ stimulation.

We performed lentiviral production using 293T cells at 70-90% confluency after allowing for incubation for 30 min at room temperature of transfection reagents including 15 μg of the EphaA3 lentiviral plasmid, 18 μg of a gag/pol/tat/rev packaging vector, 7 μg of a VSV-G envelope vector, 111 μL of the pre-complexing reagent, 129 μL of the transfection reagent, and 9.0 mL of the transfection medium before adding to the 293T cells. Then culture the transfected cells at 37° C., 5% CO2. We then harvested the cell media supernatant and concentrated by ultracentrifugation at 112,700×g for 2 h.

Human T-cells were isolated via a negative selection magnetic bead kit from peripheral blood mononuclear cells (PBMCs) from de-identified normal donor blood cones collected during apheresis. The isolated T cells were then stimulated with magnetic CD3/CD28 beads at a ratio of 3:1 beads:T cells and incubated for 24 hours.

We transduced stimulated T cells with harvested virus at a multiplicity of infection (MOI) of 3.0. CAR-T cells were then expanded by incubation at 37° C., 5% CO2, counted and fed at days 3 and 5 post lentiviral transduction and maintained at a CAR-T cell concentration of 1×10⁶/mL. Six days after transduction cell surface expression of the CAR was assessed by flow cytometry. 100,000 T cells from the culture were washed with flow buffer prepared with Dulbecco's phosphate-buffered saline, 2% fetal bovine serum, and 1% sodium azide and subsequently stained with anti-CAR antibody (goat anti-mouse was used) and washed twice. The cells were stained with live/dead stain and CD3 monoclonal antibody. The cells were washed and resuspend in flow buffer and subsequently analyzed by flow cytometry to determine transduction efficiency. For killing assays, patient derived GBM xenograft cell lines (gift from Jann Sarkaria's lab) with varying expression of the target antigen by RNA-seq analysis were incubated at the indicated ratios with effector T cells for 24 hours. Killing was calculated by bioluminescence imaging on a Xenogen IVIS-200 Spectrum camera as a measure of residual live cells. Samples were treated with 1 ul D-luciferin (30 ug/mL) per 100 ul sample volume for 10 minutes prior to imaging.

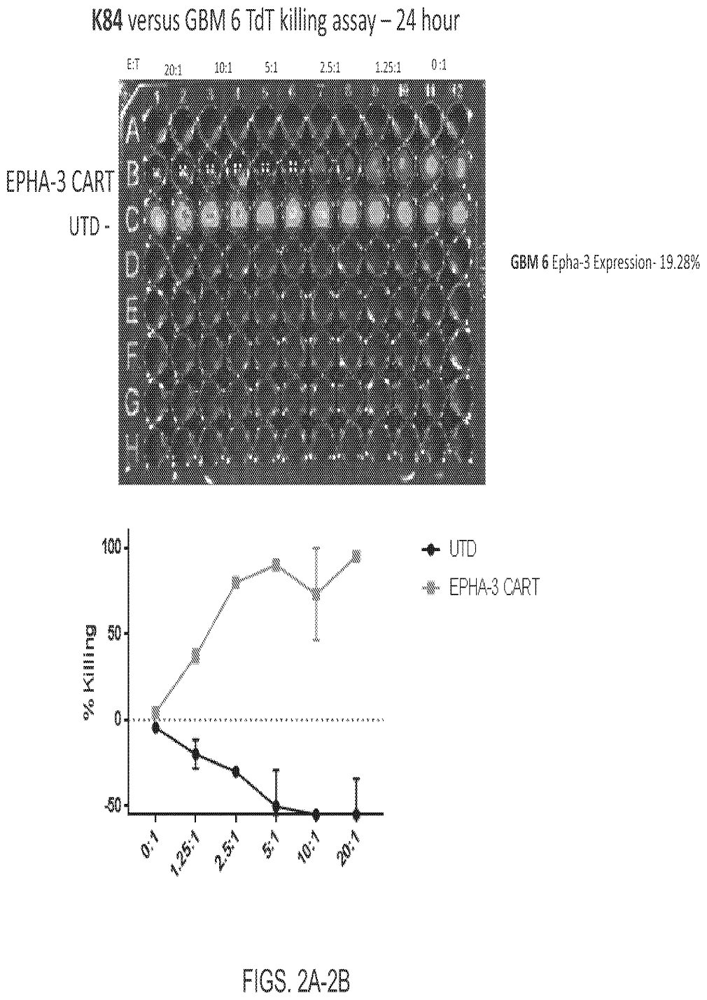

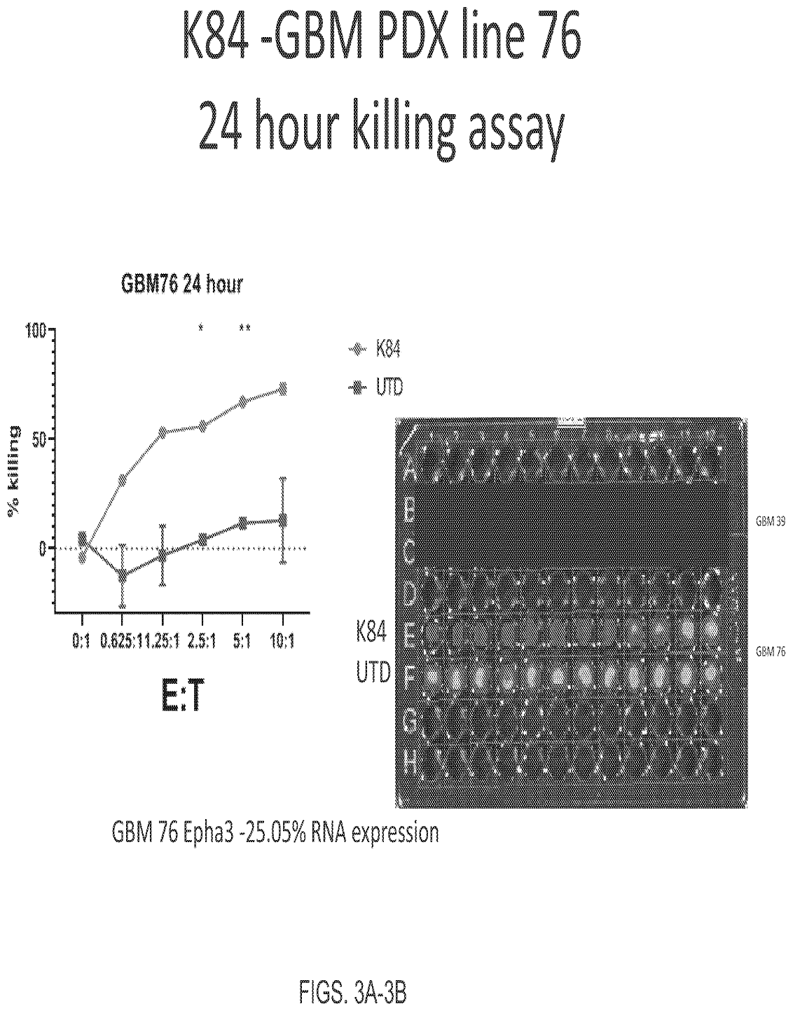

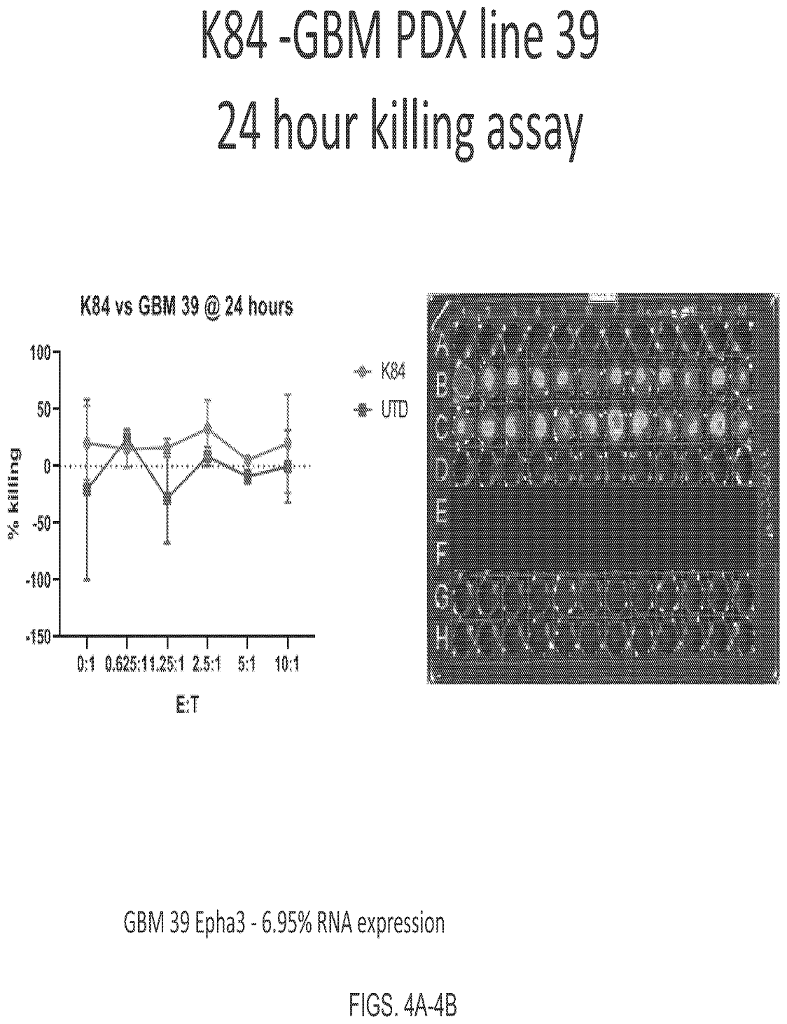

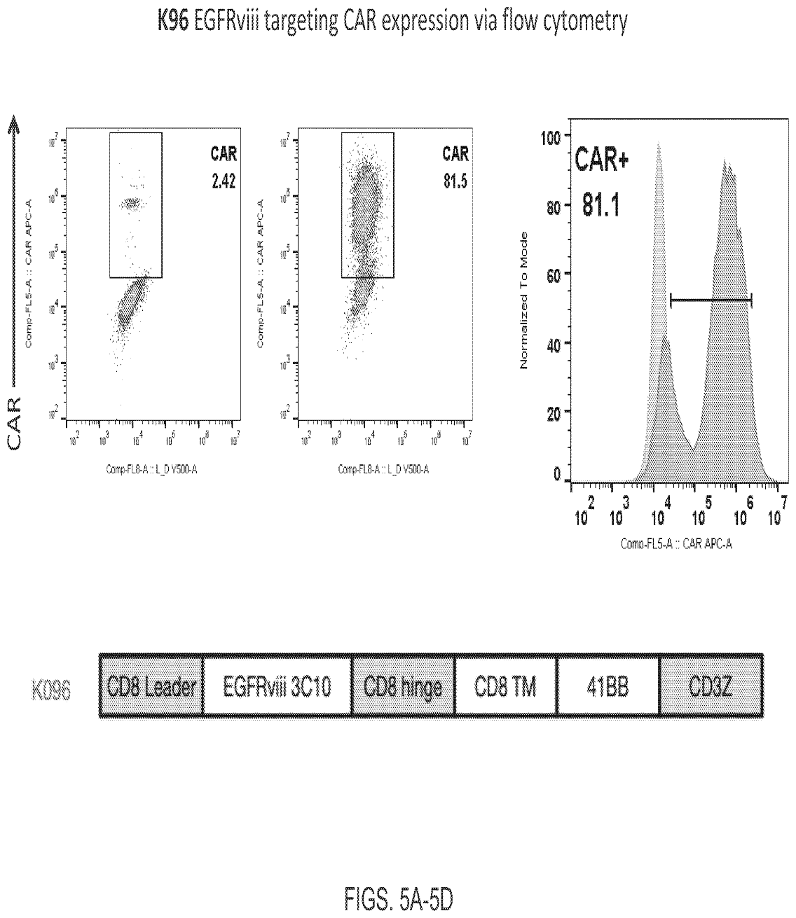

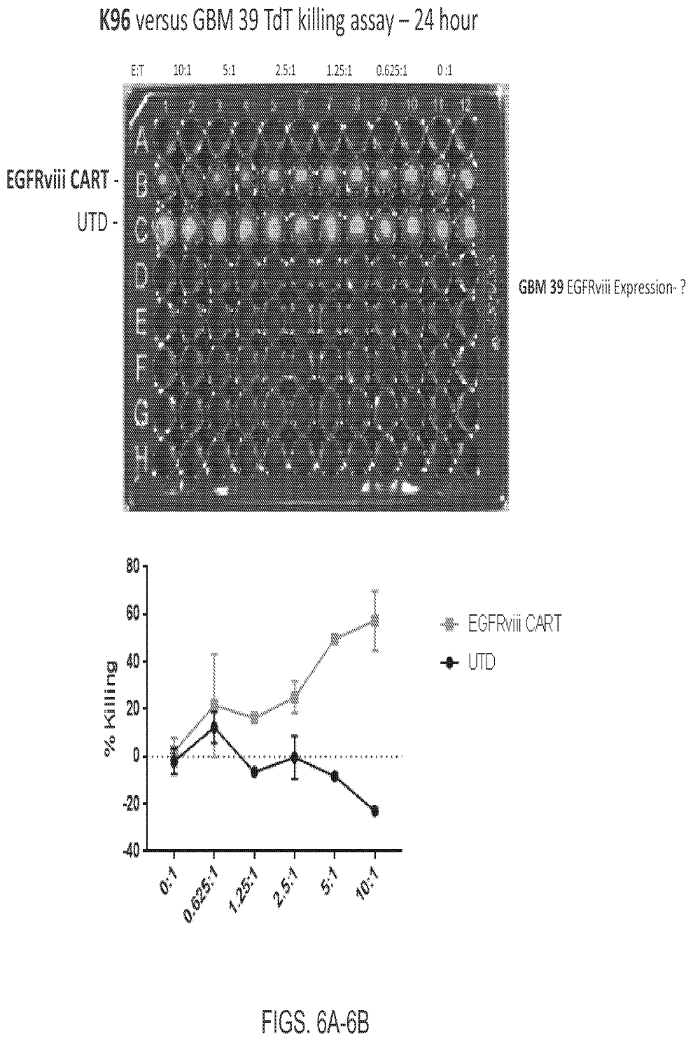

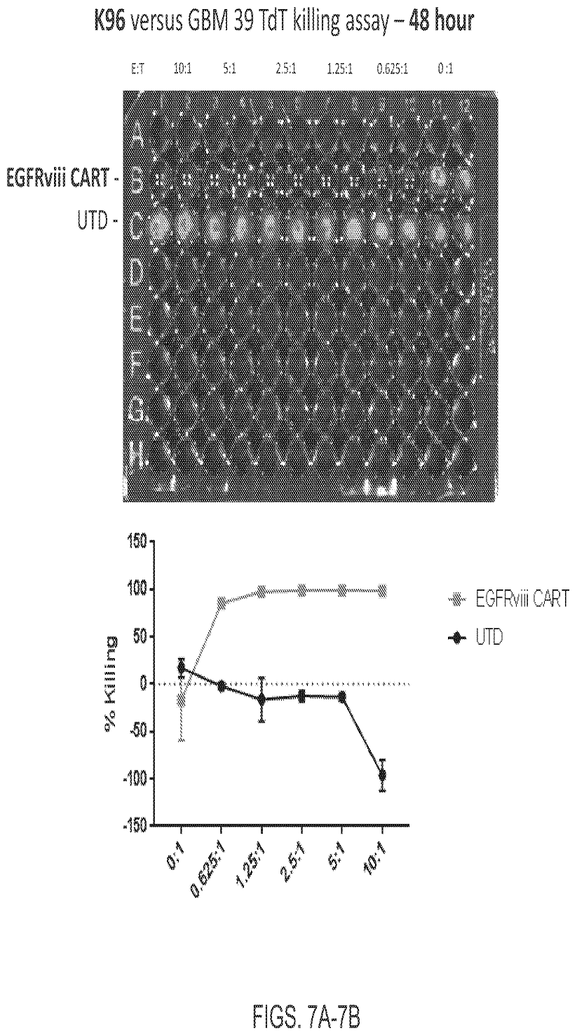

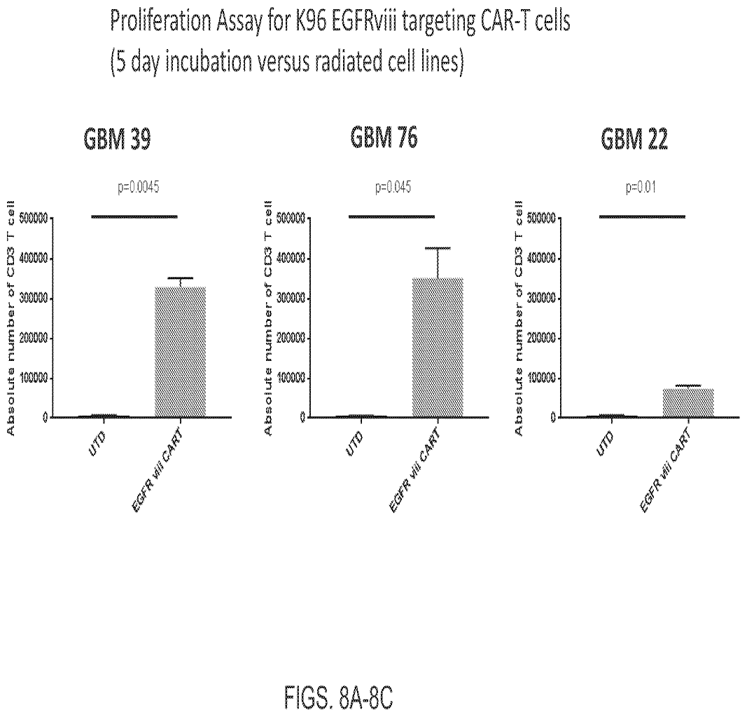

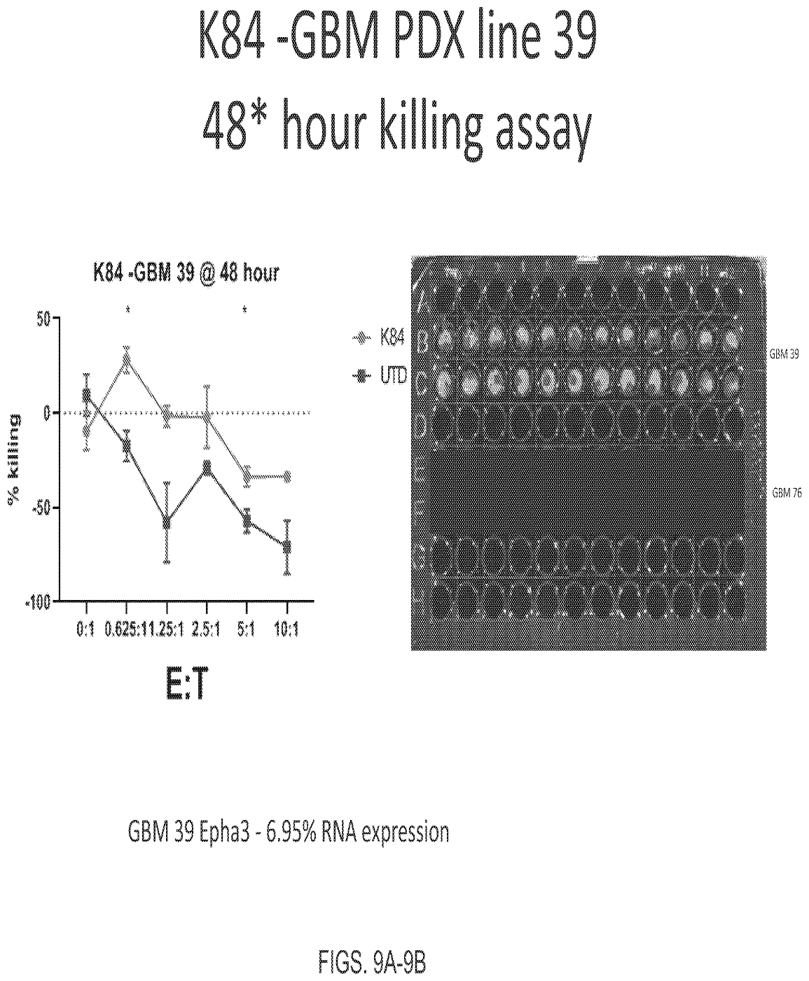

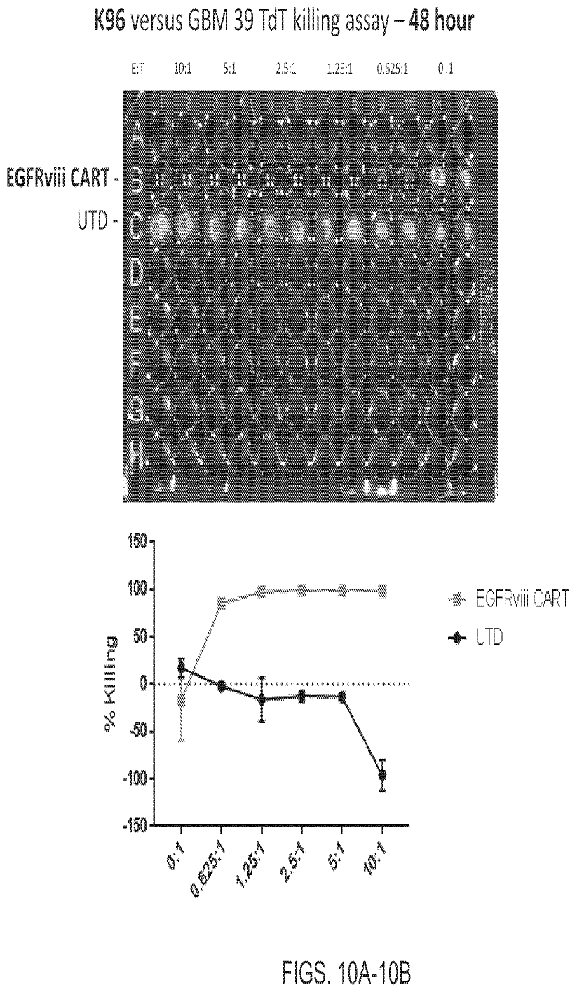

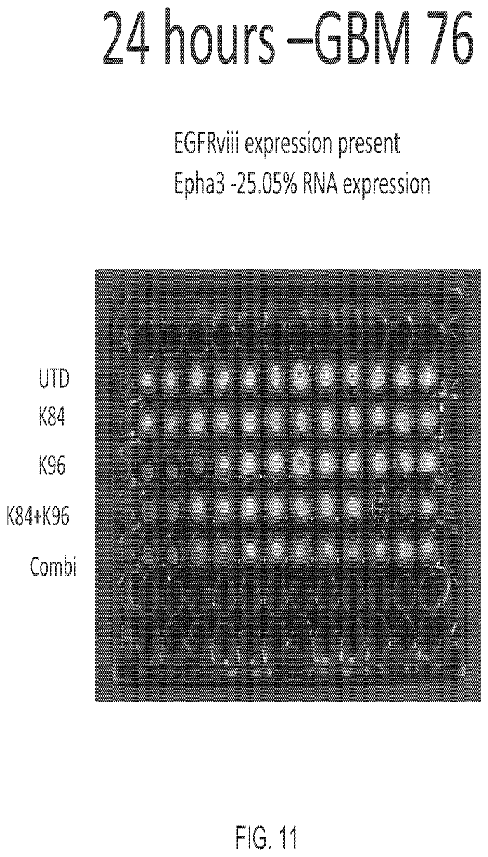

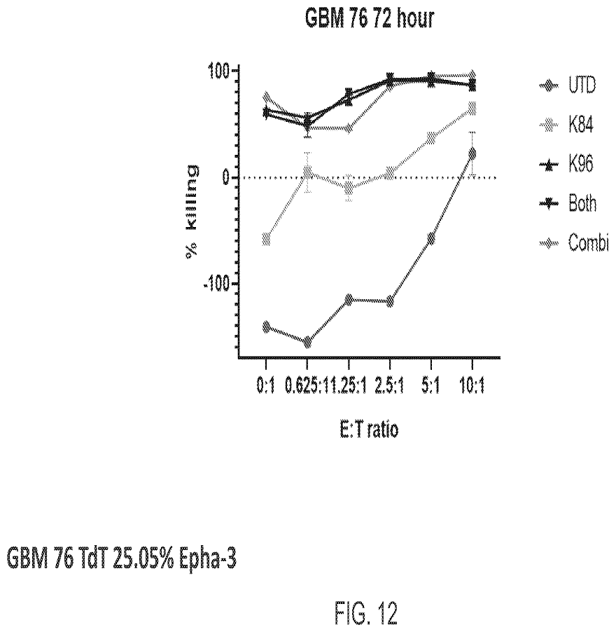

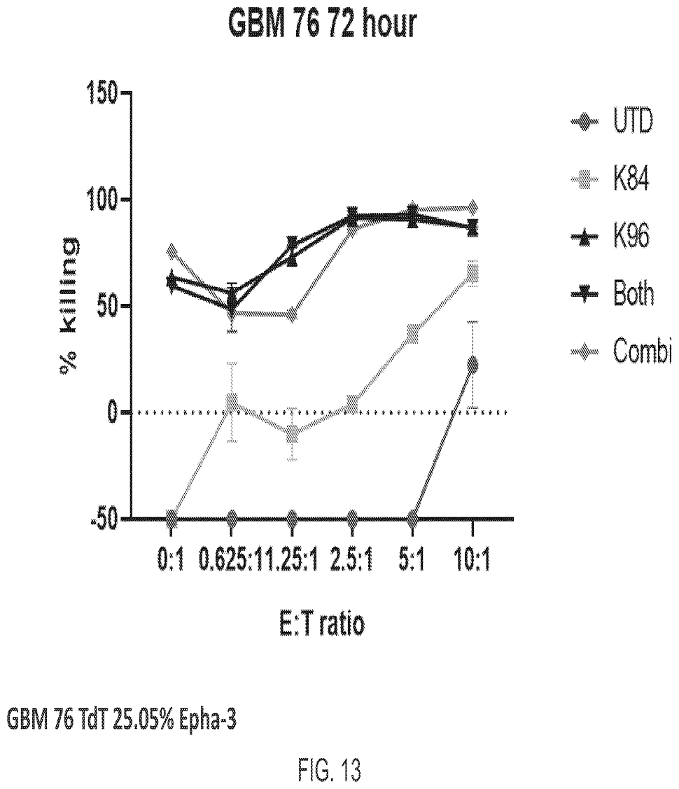

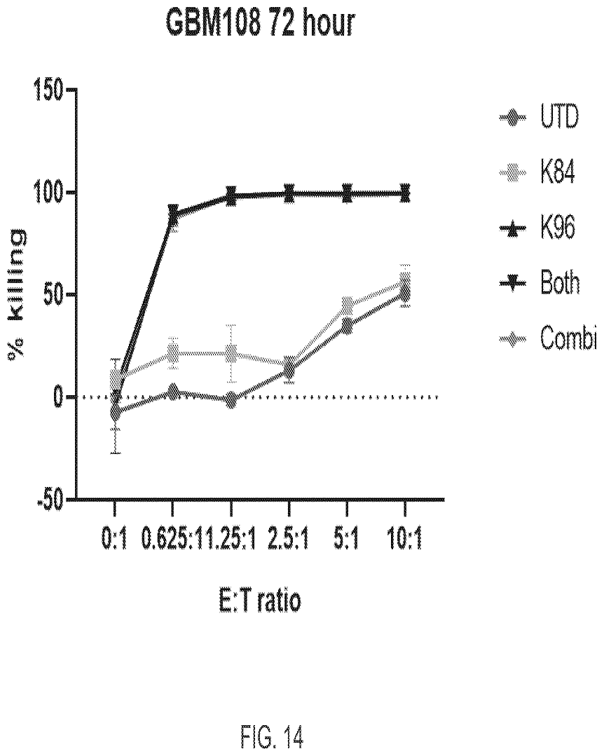

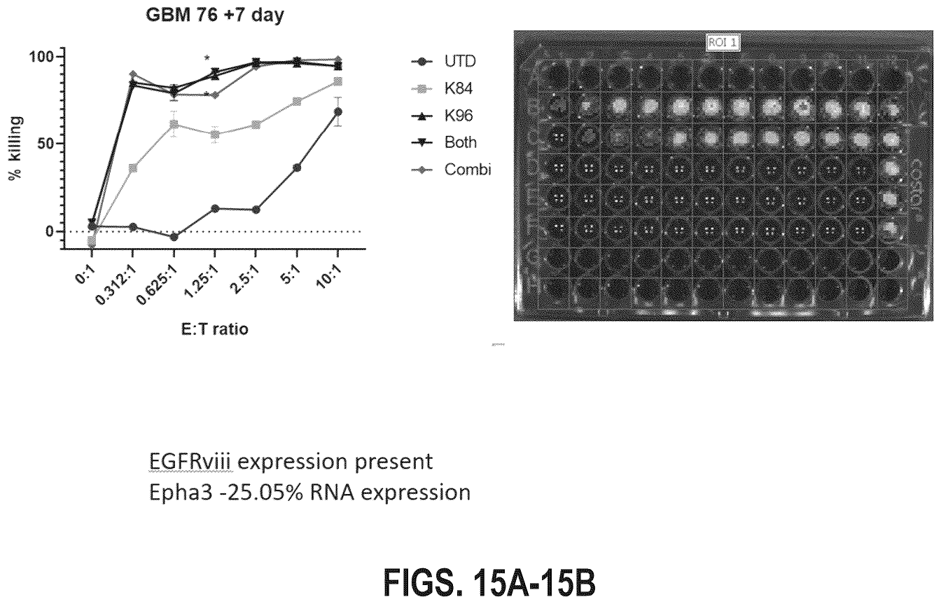

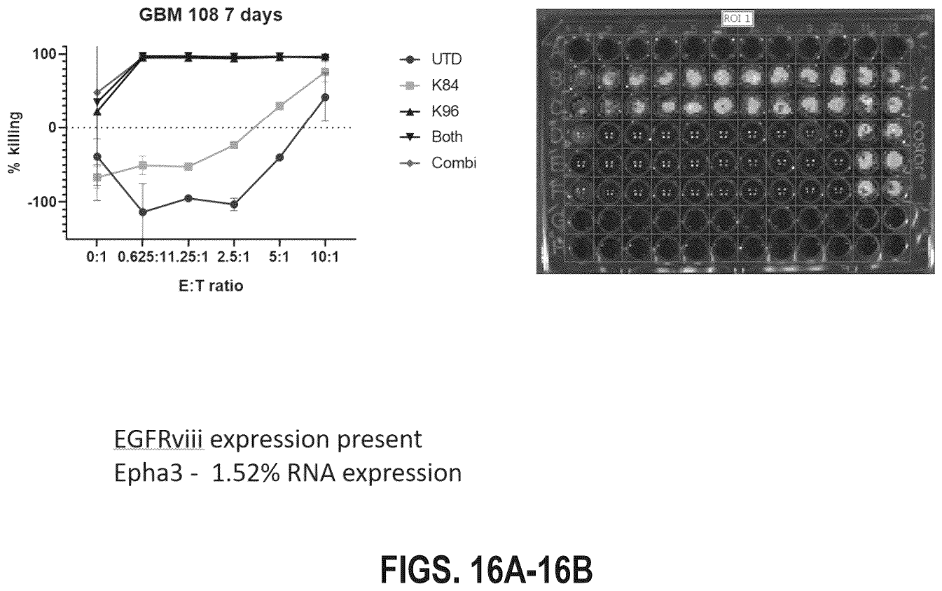

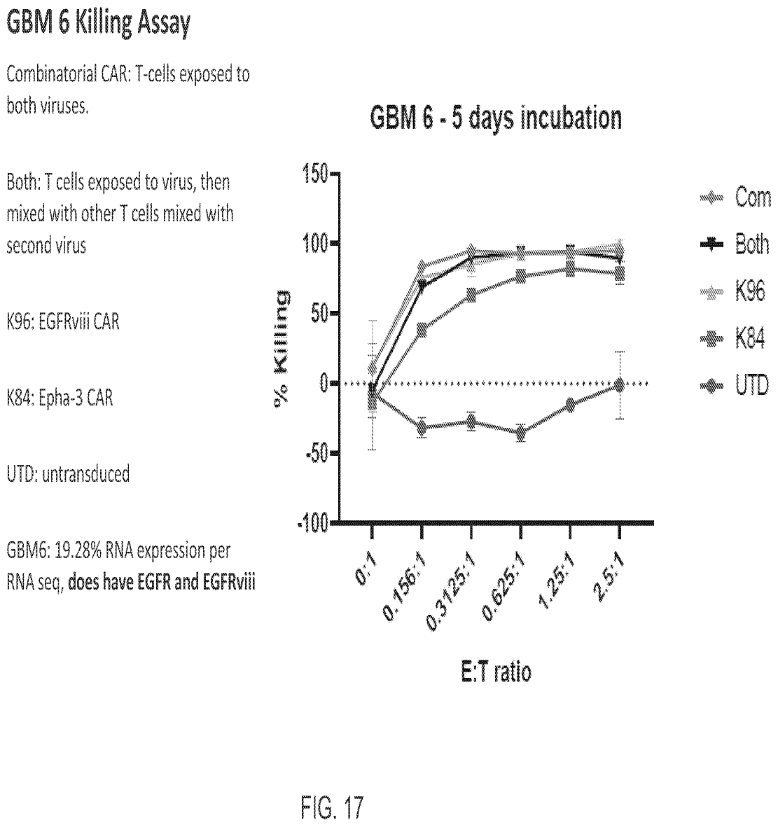

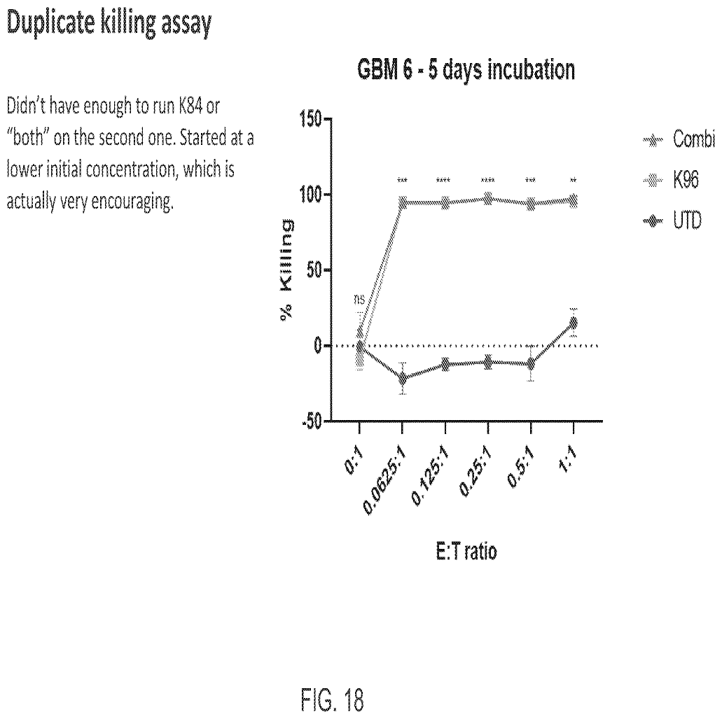

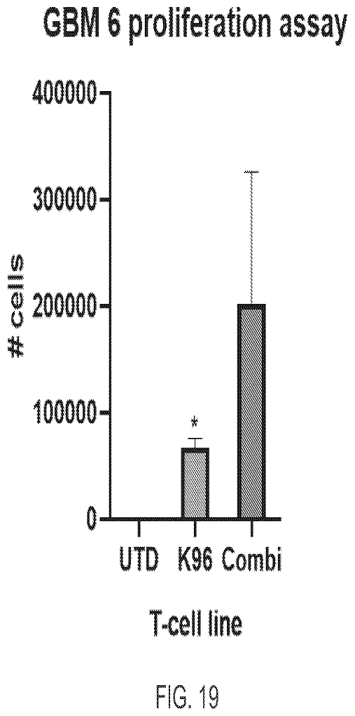

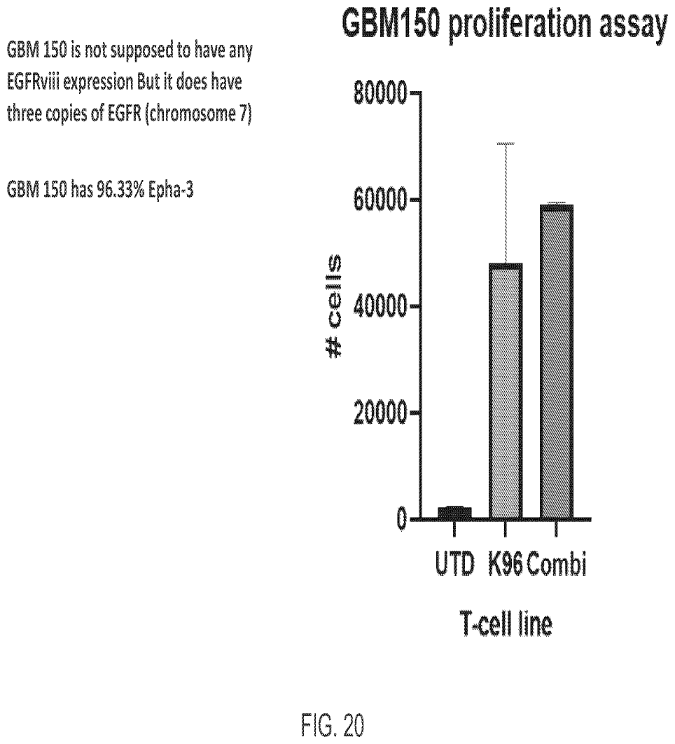

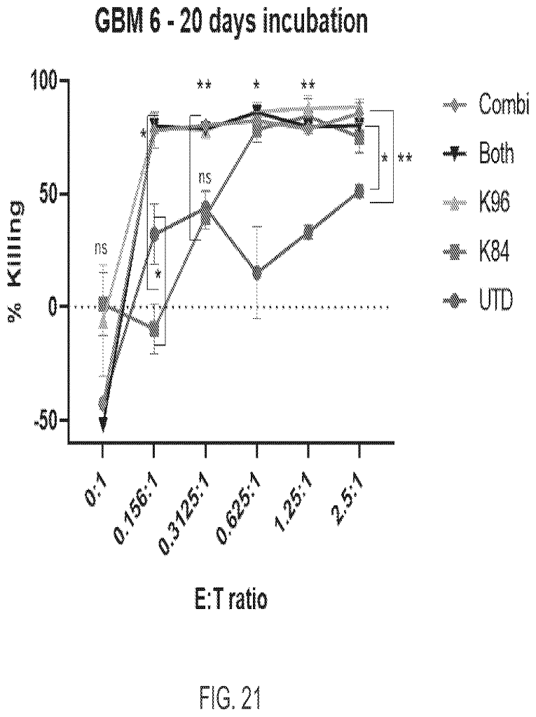

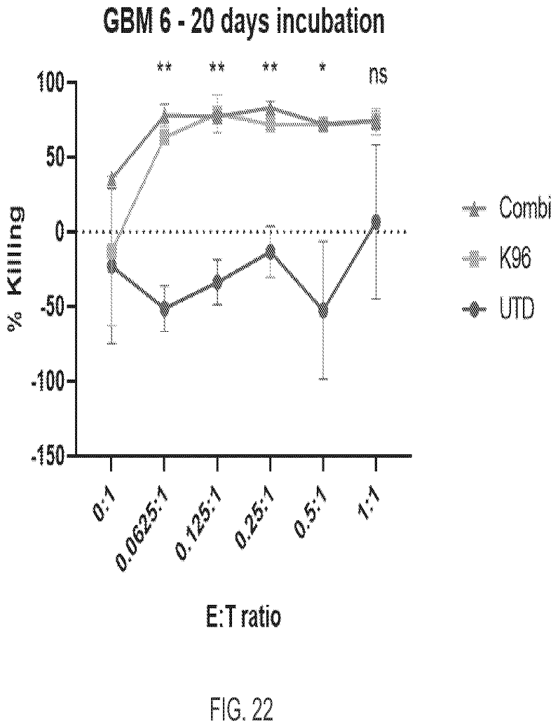

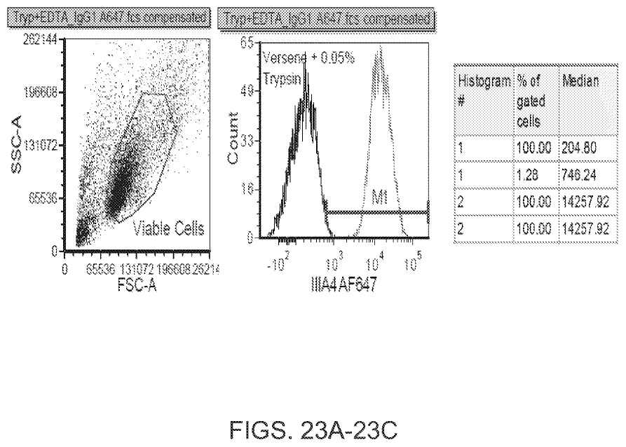

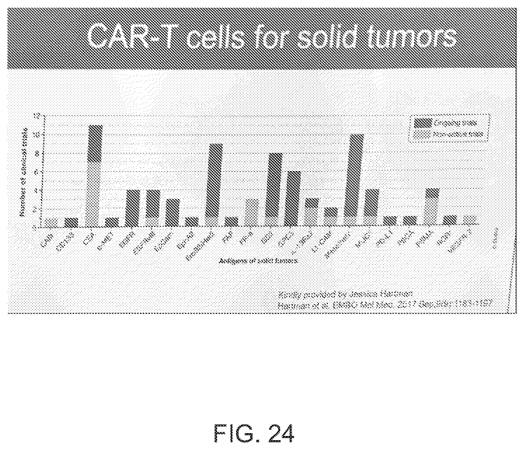

RESULTS: We demonstrated CAR-T cell production of the EphA3 targeting CAR (termed CAR K084 (or “K84”) (L2H-28z) (SEQ ID NO: 18)) on the surface of donor T-cells (FIGS. 1A-1B) by flow cytometry. A map of the CAR construct is shown (FIG. 1C). EphA3 directed CART cells exhibited specific and potent antitumor activity against EphA3+ GBM cell lines with variable transcriptome EphA3 expression indicating its broader applicability in patients with GBM (FIGS. 2B, 3A). Killing over 24-hour incubation was significant at low effector:target ratio: 52.5% killing at 1.25:1 against cell lines with 25.05% EphA3 expression and 37.1% killing at 1.25:1 and 90% killing at 5:1 ratio against a cell line with 19.28% expression. Conversely, when co-cultured with UTD controls there was significantly lower killing, or growth of tumor cells. Cell killing is demonstrated by decreased bioluminescence—a surrogate for viable tumor cells that express luciferase—when exposed to higher ratios of effector (CAR-T cell) to target (tumor cell) ratios when compared to un-transduced (UTD) from the same donor apheresis cone. There was not a significant difference in killing when co-cultured with cell lines with 6.95% EphA3 expression (FIG. 4A).

### Example 2B

METHODS: We developed a second generation CD28 co-stimulated CAR construct in a third generation lentivirus backbone to generate EphA3 CART cells using the single chain variable fragment of ifabotuzumab, a monoclonal antibody directed against EphA3. Patient derived GBM xenograft cell lines were used in these experiments. The EphA-3 CART construct was designed and then synthesized de novo using a commercially available protein synthesis vendor using the following DNA sequence comprising a CD8 leader, a single chain variable fragment (scFv) of anti-human EphA3 monoclonal antibody ifabotuzumab (Ifab scFv) and a CD8 hinge:

(SEQ ID NO: 55)
GCTAGCTCTAGAATGGCCTTACCAGTGACCGCCTTGCTCCTGCCGCTGGC
CTTGCTGCTCCACGCCGCCAGGCCGGACATCCAGATGACCCAGTCTCCAT
CCTTCCTGTCTGCATCTGTAGGAGACAGAGTCACCATCACTTGCCGGGCC
AGTCAGGGCATTATCAGTTATTTAGCCTGGTATCAGCAAAAACCAGAGAA
AGCCCCTAAGCGCCTGATCTATGCTGCATCCAGTTTGCAAAGTGGGGTCC
CATCAAGGTTCAGCGGCAGTGGATCTGGGACAGAATTCACTCTCACAATC
AGCAGCCTGCAGCCTGAAGATTTTGCAACTTACTACTGCGGGCAGTATGC
CAATTATCCGTACACCTTTGGCCAAGGTACGAAACTGGAAATTAAAGGTG
GAGGTGGTTCGGGAGGTGGAGGTAGCGGAGGTGGTGGATCTCAGGTGCAG
CTGGTGCAGTCTGGGGCTGAGGTGAAGAAGCCTGGGGCCTCAGTGAAGGT
TTCCTGCAAGGCTTCTGGATACACCTTCACAGGCTACTGGATGAATTGGG
TGCGCCAGGCCCCCGGGCAAGGCCTGGAGTGGATGGGGGACATCTACCCG
GGCAGTGGTAACACAAACTACGATGAGAAGTTCCAGGGTAGAGTCACGAT
GACCAGGGACACGTCCATCAGCACAGCCTACATGGAGCTGAGCAGGCTGA
GATCTGACGACACAGCCGTGTACTACTGCGCAAGAGGTGGATATTATGAA
GATTTTGATAGCTGGGGCCAAGGTACCACTGTGACCGTGAGCTCCCTCGA
GCCCAAATCTTGTGACAAAACTCACACATGCCCACCGTGCCCGGATCCCA
AATTTTGGGTGCTGGTGGTGGTTGGTGGAGTCCTGGCTTGCTATAGCTTG
CTAGTAACAGTGGCCTTTATTATTTTCTGGGTGAGGAGTAAGAGGAGCAG
GCTCCTGCACAGTGACTACATGAACATGACTCCCCGCCGCCCCGGGCCCA
CCCGCAAGCATTACCAGCCCTATGCCCCACCACGCGACTTCGCAGCCTAT
CGCTCCAGAGTGAAGTTCAGCAGGAGCGCAGACGCCCCCGCGTACAAGCA
GGGCCAGAACCAGCTCTATAACGAGCTCAATCTAGGACGAAGAGAGGAGT
ACGATGTTTTGGACAAGAGACGTGGCCGGGACCCTGAGATGGGGGGAAAG
CCGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGCTAAGTCGA
C.

The EphA-3 CAR construct was subsequently cloned into a third generation lentivirus under control of an EF-1α promoter. The single chain variable region fragment is a proprietary sequence produced by Humanigen. The EphA-3 construct possesses a second generation CD28 costimulatory domain and CD3ζ stimulation.

We performed lentiviral production using 293T cells at 70-90% confluency after allowing for incubation for 30 min at room temperature of transfection reagents including 15 μg of the Epha-3 lentiviral plasmid, 18 μg of a gag/pol/tat/rev packaging vector, 7 μg of a VSV-G envelope vector, 111 μL of the pre-complexing reagent, 129 μL of the transfection reagent, and 9.0 mL of the transfection medium before adding to the 293T cells. Then culture the transfected cells at 37° C., 5% CO2. We then harvested the cell media supernatant and concentrated by ultracentrifugation at 112,700×g for 2 h.

Human T-cells were isolated via a negative selection magnetic bead kit from peripheral blood mononuclear cells (PBMCs) from de-identified normal donor blood cones collected during apheresis. The isolated T cells were then stimulated with magnetic CD3/CD28 beads at a ratio of 3:1 beads:T cells and incubated for 24 hours.

We transduced stimulated T cells with harvested virus at a multiplicity of infection (MOI) of 3.0. CAR-T cells were then expanded by incubation at 37° C., 5% CO2, counted and fed at days 3 and 5 post lentiviral transduction and maintained at a CAR-T cell concentration of 1×10\hat{ }6/mL. Six days after transduction cell surface expression of the CAR was assessed by flow cytometry. 100,000 T cells from the culture were washed with flow buffer prepared with Dulbecco's phosphate-buffered saline, 2% fetal bovine serum, and 1% sodium azide and subsequently stained with anti-CAR antibody (goat anti-mouse was used) and washed twice. The cells were stained with live/dead stain and CD3 monoclonal antibody. The cells were washed and resuspend in flow buffer and subsequently analyzed by flow cytometry to determine transduction efficiency. For killing assays, patient derived GBM xenograft cell lines (gift from Jann Sarkaria's lab) with varying expression of the target antigen by RNA-seq analysis were incubated at the indicated ratios with effector T cells for 24 hours. Killing was calculated by bioluminescence imaging on a Xenogen IVIS-200 Spectrum camera as a measure of residual live cells. Samples were treated with 1 ul D-luciferin (30 ug/mL) per 100 ul sample volume for 10 minutes prior to imaging.

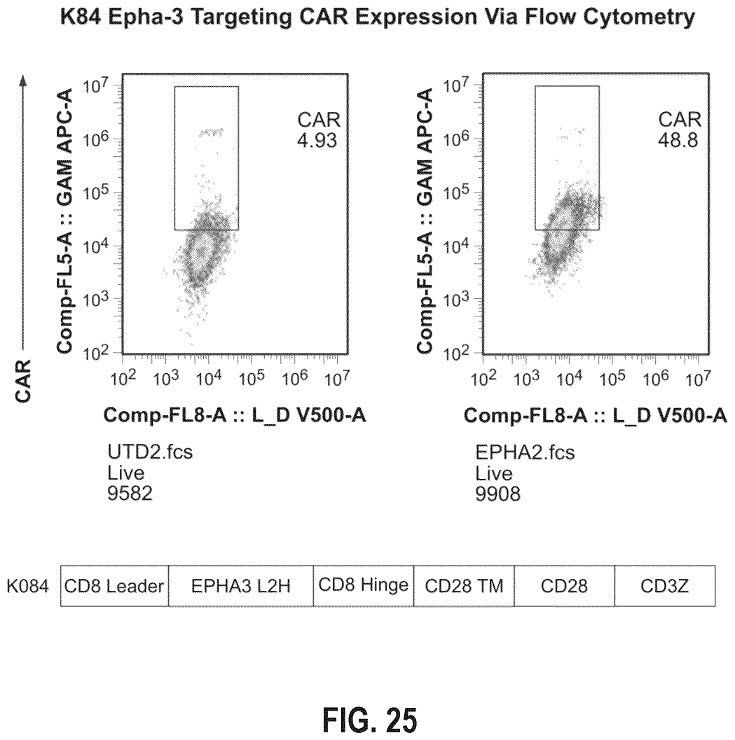

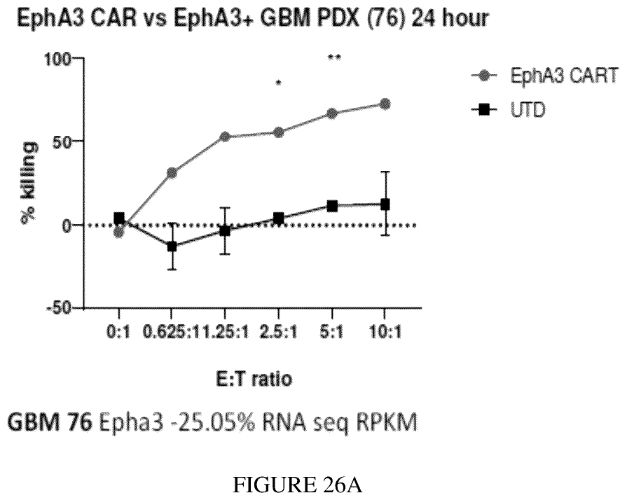

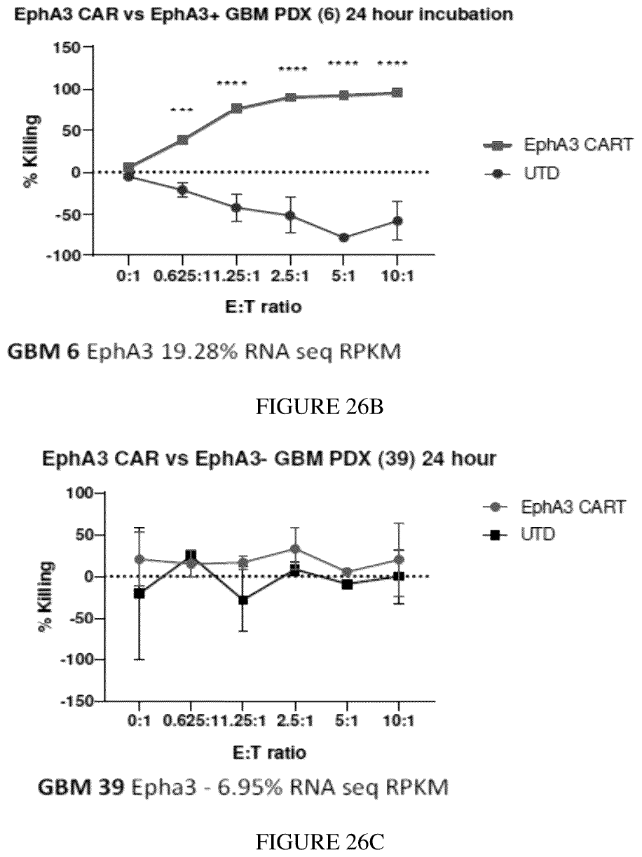

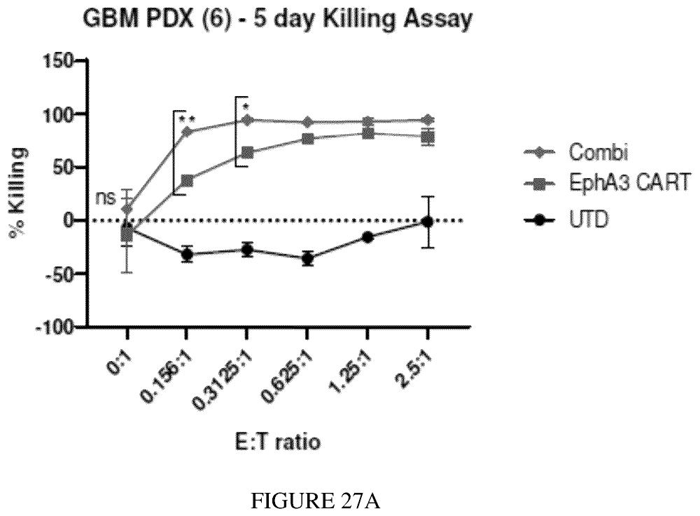

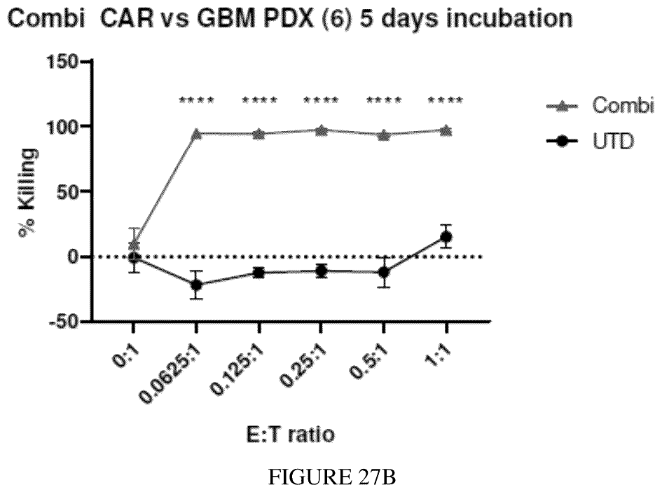

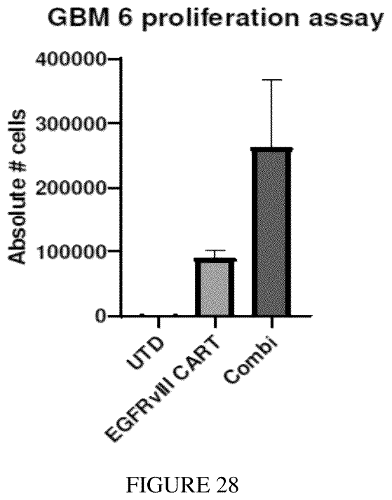

RESULTS: We demonstrated CAR-T cell production of the Epha-3 targeting CAR (termed K84) on the surface of donor T-cells (FIG. 1) by flow cytometry. A map of the CAR construct is shown (FIG. 1). EphA3 directed CART cells exhibited specific and potent antitumor activity against EphA3+ GBM cell lines with variable transcriptome EphA3 expression indicating its broader applicability in patients with GBM (FIG. 2). Killing over 24-hour incubation was significant at low effector:target ratio: 52.5% killing at 1.25:1 against cell lines with 25.05% EphA3 expression and 37.1% killing at 1.25:1 and 90% killing at 5:1 ratio against a cell line with 19.28% expression. Conversely, when co-cultured with UTD controls there was significantly lower killing, or growth of tumor cells. Cell killing is demonstrated by decreased bioluminescence—a surrogate for viable tumor cells that express luciferase—when exposed to higher ratios of effector (CAR-T cell) to target (tumor cell) ratios when compared to un-transduced (UTD) from the same donor apheresis cone. There was not a significant difference in killing when co-cultured with cell lines. FIG. 25 shows EphA3 CAR expression on transduced T-cells (right) compared to untransduced T-cells (Left); a construct map is shown at the bottom. FIGS. 26A-26C show EphA3 CAR bioluminescence killing assay at 24 hour incubation versus GBM 76, GBM 6 and GBM 39. FIGS. 27A-27B show dual EPHA3/EGFRvIII targeting CART cells exhibit potent antitumor killing against patient derived GBM. In FIG. 28, combinatorial EGFRvIII and EphA3 CARs demonstrate increased proliferation when exposed to target antigen.

Conclusion: We demonstrate for the first time that targeting EphA3 with CART cells is feasible, specific, and efficacious and represents a novel therapeutic strategy to target GBM.

### Example 3

### Generation of CAR Binding to Human EphA3 and CART Cells Comprising the CAR

A CAR construct in lentivirus backbone is designed and synthesized as described in Example 1 to generate EphA3 CART cells comprising a chimeric antigen receptor (CAR) construct that binds to human Eph receptor A3 (EphA3) using a human EphA3 single chain variable fragment (scFv) of anti-EphA3 monoclonal antibody, wherein the human EphA3 scFv comprises CDR3 of the V_{H} region comprises GGYYEDFDS (SEQ ID NO:44) and the CDR3 of the V_{L} region comprises GQYANYPYT (SEQ ID NO:45).

### Example 4

### Generation of CAR Binding to Human EphA3 and CART Cells Comprising the CAR

A CAR construct in lentivirus backbone is designed and synthesized as described in Example 1 to generate EphA3 CART cells comprising a chimeric antigen receptor (CAR) construct that binds to human Eph receptor A3 (EphA3) using an extracellular anti-human EphA3 binding domain comprising:
a V_{H} region comprising amino acid sequence:
   QVQLVQSGAEVKKPGASVKVSCKASGYTFTGYWMNWVRQAPGQGLEWMGDIY PGSGNTNYDEKFQGRVTMTRDTSISTAYMELSRLRSDDTAVYYCARGGYYEDFD SWGQGTTVTVSS (SEQ ID NO:46) and
a V_{L} region comprising amino acid sequence:
   DIQMTQSPSFLSASVGDRVTITCRASQGIISYLAWYQQKPEKAPKRLIYA ASSLQSGVPSRFSGSGSGTEFTLTISSLQPEDFATYYCGQYANYPYTFGQ GTKLEIK (SEQ ID NO:47).

### Example 5

### Generation of CAR Binding to Human EphA3 and CART Cells Comprising the CAR

A CAR construct in lentivirus backbone is designed and synthesized as described in Example 1 to generate EphA3 CART cells comprising a chimeric antigen receptor (CAR) construct that binds to human Eph receptor A3 (EphA3) using an extracellular anti-human EphA3 binding domain comprising:
a V_{H} region CDR1 having a sequence SYWIN (SEQ ID NO:48), a V_{H} region CDR2 having a sequence DIYPGSGNTNYDEKFKR (SEQ ID NO:49), a V_{H} region CDR3 having a sequence SGYYEDFDS (SEQ ID NO:50), and
a V_{L} region CDR1 having a sequence RASQEISGYLG (SEQ ID NO:51), a V_{L} region CDR2 having a sequence AASTLDS (SEQ ID NO:52), and a V_{L} region CDR3 having a sequence VQYANYPYT (SEQ ID NO:53).

### Example 6

### Combination Therapy

A subject having a tumor that expresses EphA3 on its cell surface, or on the surface of tumor associated cells, is treated with human EphA3-targeting CAR T-cells, as described herein, and at least one immune checkpoint inhibitor, such as an anti-CTLA-4 antibody and/or an anti-PD-1 antibody to enhance the anti-tumor activity of the human EphA3-targeting CAR T-cells. The human EphA3-targeting CAR T-cells and the anti-CTLA-4 antibody and/or the anti-PD-1 antibody are administered sequentially in any order. CART cells (108-109) are dosed every 2-3 days for 16-20 days. Anti-CTLA-4 antibody and/or anti-PD-1 antibody are dosed monthly.

A tumor expressing EphA3 on its cell surface or the surface of tumor associated cells may be reduced in size by >50% within 3 months. After 12 months (4 cycles) of administration of this combination therapy, the subject's tumor may not be detectable by an imaging technique, such as a radiograms, a CT scan, an MRI, a PET scan and/or an ultrasound.

### Example 7

### Combination Therapy

A subject having a tumor that expresses EphA3 on its cell surface, or on the surface of tumor associated cells, is treated with human EphA3-targeting CAR T-cells, as described herein, and at least one immune checkpoint inhibitor, such as an anti-CTLA-4 antibody and/or an anti-PD-1 antibody to enhance the anti-tumor activity of the human EphA3-targeting CAR T-cells. The human EphA3-targeting CAR T-cells and the anti-CTLA-4 antibody and/or the anti-PD-1 antibody are administered sequentially in any order. If split dosing is required due to toxicity issues, the dosages of CART cells are split into two separate doses between day 0 and day 5 or 7. The anti-CTLA-4 antibody and/or anti-PD-1 antibody are dosed Q3 weeks.

### Example 8

### EphA3-Targeting CAR T-Cell and Immune Checkpoint Inhibitor Combination Therapy

In solid tumor patients receiving CAR-T therapy better clinical outcomes have been observed in those in which the CAR-T cells persist in the blood beyond 6 weeks. This suggests that clinical efficacy of CAR-T cells depends on their persistence. Suppression or exhaustion of CAR-T cells can lead to reduced expansion and persistence and hence reduced effect. This can be caused by PD-1/PD-L1 signaling which attenuates T-cell activities after antigen binding. PD-1 can be up-regulated on CART-cells in vivo leading to a loss in efficacy. It has been shown by others that the activity of tumor infiltrating T-cells can be inhibited by the up-regulation of both CTLA-4 and PD-1 co-inhibitory signals. Antibody blockade of CTLA-4 and/or PD-1 can enhance anti-tumor activity.

Ifabotuzumab is a humaneered monoclonal antibody that binds to human and mouse EphA3 receptor. A second generation CD28 co-stimulated and CD3ζ stimulated, anti-EphA3 CAR-T construct using the single chain variable region fragment of ifabotuzumab was constructed in a third generation lentivirus vector backbone (as described in this application).

Human small cell lung cancer cell line NCI-H446 expresses EphA3 on its cell surface as demonstrated by Flow cytometry studies using mouse anti-EphA3 antibody IIIA4 conjugated to Alexa Fluor 647 dye (FIGS. 23A-23C).

NCI-H446 cells are cultured in complete DMEM medium in flasks until they cover 70% of the bottom. Cells are then digested with 0.25% trypsin and passaged for expansion. Finally, the cancer cells are harvested, and then 1×10⁶ cells injected subcutaneously into the left front dorsum of immune-deficient, NSG mice. Mice are monitored and subcutaneous (sc) tumors growth assessed. When tumors measure approximately 100 mm² dosing begins. Mice receive 1-1.5×10⁶ CART cells (either transduced and expressing anti-EphA3 TCR or untransduced) intravenously. One group of mice also receives anti-PD1 monoclonal antibody (10 mg/kg) IP on day 0 and then every third day. Another group received anti-CTLA4 antibody (10 mg/kg) dosed every third day. Dosing continues for 25 days or until mice are terminated due to tumors reaching 1500 mm². Anti-tumor effects of EphA3-CART alone and in combination with anti-PD1 and/or anti-CTLA4 are assessed by measuring tumor size and overall survival.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. method for treating a solid tumor cancer, the method comprising administering to a subject in need thereof a pharmaceutical composition comprising:
a human Eph receptor A3 (EphA3)-targeting chimeric antigen receptor (CAR)-T cell comprising a human EphA3-targeting CAR expression cassette comprising a CAR that binds to human EphA3, the CAR comprising an extracellular anti-human EphA3 binding domain comprising a single chain variable fragment (scFv) of an anti-human EphA3 monoclonal antibody, wherein the human EphA3 scFv comprises a heavy chain immunoglobulin variable region (V_{H}) Comprising an amino acid sequence of SEQ ID NO: 1 and a light chain immunoglobulin variable region (V_{L}) comprising an amino acid sequence of SEQ ID NO: 2, wherein the extracellular anti-human EphA3 binding domain is connected to a CD28 transmembrane domain by a CD28 hinge region, wherein the CAR further comprises an intracellular signaling domain, wherein the intracellular signaling domain comprises a costimulatory domain and an activation domain, wherein the costimulatory domain comprises a CD28 costimulatory domain, wherein the activation domain is a CD3 zeta (CD3z) or CD3Q activation domain, and wherein the CAR is expressed in the T cell, cloned into a lentivirus backbone under control of a promoter, wherein the CAR-T cell expresses the CAR on a cell surface thereof; and
a pharmaceutically acceptable carrier,
wherein the EphA3-targeting CAR T-cell is an autologous EphA3-targeting CAR T-cell.

2. The method of claim 1, wherein the solid tumor cancer is a brain cancer, wherein the brain cancer is glioblastoma multiforme (GBM):
wherein the solid tumor cancer is a colon, lung, kidney, bladder, breast, liver, pancreatic, prostate, a melanoma, myeloma, wherein the lung cancer is a non-small cell lung cancer (NSCLC) or small cell lung cancer (SCLC);
wherein the solid tumor cancer expresses EphA3 on a surface of tumor cells, tumor-associated vasculature and/or tumor stroma cells; wherein the tumor stroma cells comprise myeloid derived suppressor cells; wherein the tumor cells comprise tumor stem cells and/or wherein the tumor-associated vasculature comprises neo-vasculature.

3-8. (canceled)

9. The method of claim 1, further comprising administering a human GM-CSF (hGM-CSF) antagonist selected from the group consisting of an anti-human GM-CSF antibody, an anti-hGM-CSF antibody fragment, a soluble hGM-CSF receptor alpha, an anti-hGM-CSF receptor (GM-CSFr) antibody and an anti-hGM-CSFr antibody fragment, wherein the anti-human GM-CSF antibody is hGM-CSF neutralizing antibody lenzilumab, Namilumab, Otilimab, Gimsilumab, TJM2 (TJ003234) or Mavrilimumab.

10-11. (canceled)

12. The method of claim 9, wherein administration of lenzilumab reduces relapse rate or prevents occurrence of solid tumor relapse; wherein administration of lenzilumab prevents or reduces incidence of immunotherapy-related toxicity in the subject; wherein the immunotherapy-related toxicity is EphA3-targeting CAR-T cell related toxicity and/or wherein the EphA3-targeting CAR-T cell related toxicity is cytokine release syndrome, neurotoxicity and/or neuro-inflammation.

13-15. (canceled)

16. The method of claim 1, further comprising administering GM-CSF silenced EphA3-targeting CAR-T cells or gene knockout (GM-CSF^{k/o}) EphA3-targeting CAR-T cells, wherein administration of GM-CSF silenced EphA3-targeting CAR-T cells or GM-CSF^{k/o} EphA3-targeting CAR-T cells prevents or reduces incidence of immunotherapy-related toxicity in the subject:
wherein administration of the GM-CSF silenced EphA3-targeting CAR-T cells or GM-CSF^{k/o} EphA3-targeting CAR-T cells reduces relapse rate or prevents occurrence of solid tumor relapse.

17. (canceled)

18. The method of claim 1, further comprising administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody;
wherein the anti-programmed cell death-1 (PD-1) antibody is Pembrolizumab, the anti-programmed death-ligand antibody (PD-L1) is avelumab, and. the anti-cytotoxic T-lymphocyte associated protein 4 (CTLA-4) antibody is Ipilimumab.

19. (canceled)

20. The method of claim 16, further comprising administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody;
wherein the anti-programmed cell death-1 (PD-1) antibody is Pembrolizumab, the anti-programmed death-ligand antibody (PD-L1) is avelumab, and. the anti-cytotoxic T-lymphocyte associated protein 4 (CTLA-4) antibody is Ipilimumab.

21. (canceled)

22. A method for reducing solid tumor cancer relapse rate or preventing occurrence of solid tumor cancer relapse in a subject in need thereof, the method comprising administering to a subject a pharmaceutical composition comprising:
a human Eph receptor A3 (EphA3)-targeting chimeric antigen receptor (CAR)-T cell comprising a human EphA3-targeting CAR expression cassette comprising a CAR that binds to human EphA3, the CAR comprising an extracellular anti-human EphA3 binding domain comprising a single chain variable fragment (scFv) of an anti-human EphA3 monoclonal antibody, wherein the human EphA3 scFv comprises a heavy chain immunoglobulin variable region (V_{H}) Comprising an amino acid sequence of SEQ ID NO: 1 and a light chain immunoglobulin variable region (V_{L}) comprising an amino acid sequence of SEQ ID NO: 2 cloned into a lentivirus backbone under control of a promoter, wherein the CAR-T cell expresses the CAR on a cell surface thereof; and
a pharmaceutically acceptable carrier,
wherein the EphA3-targeting CAR T-cell is an autologous EphA3-targeting CAR T-cell;
wherein the solid tumor cancer is a brain cancer, wherein the brain cancer is glioblastoma multiforme (GBM);
wherein the solid tumor cancer is a colon, lung, kidney, bladder, breast, liver, pancreatic, prostate tumor, a melanoma, myeloma, wherein the lung cancer is a NSCLC or a SCLC;
wherein the solid tumor cancer expresses EphA3 on a surface of tumor cells, tumor-associated vasculature and/or tumor stroma; wherein the tumor stroma cells comprise myeloid derived suppressor cells; wherein the tumor cells comprise tumor stem cells and/or wherein the tumor-associated vasculature comprises neo-vasculature.

23-29. (canceled)

30. The method of claim 22, further comprising administering a human GM-CSF (hGM-CSF) antagonist selected from the group consisting of an anti-human GM-CSF antibody, an anti-hGM-CSF antibody fragment, a soluble hGM-CSF receptor alpha, an anti-hGM-CSF receptor (GM-CSFr) antibody and an anti-hGM-CSFr antibody fragment, wherein the anti-human GM-CSF antibody is hGM-CSF neutralizing antibody lenzilumab, Namilumab, Otilimab, Gimsilumab, TJM2 (TJ003234) or Mavrilimumab.

31-32. (canceled)

33. The method of claim 31, wherein administration of lenzilumab reduces relapse rate or prevents occurrence of solid tumor relapse; wherein administration of lenzilumab prevents or reduces incidence of immunotherapy-related toxicity in the subject; wherein the immunotherapy-related toxicity is EphA3-targeting CAR-T cell related toxicity; and/or wherein the EphA3-targeting CAR-T cell related toxicity is cytokine release syndrome, neurotoxicity and/or neuro-inflammation.

34-36. (canceled)

37. The method of claim 22, further comprising administering GM-CSF silenced EphA3-targeting CAR-T cells or gene knockout (GM-CSF^{k/o}) EphA3-targeting CAR-T cells, wherein administration of GM-CSF silenced EphA3-targeting CAR-T cells or GM-CSF^{k/o} EphA3-targeting CAR-T cells prevents or reduces incidence of immunotherapy-related toxicity in the subject.

38. The method of claim 22, further comprising administering at least one immune checkpoint inhibitor selected from the group consisting of an anti-programmed cell death-1 (PD-1) antibody, an anti-programmed death-ligand 1 (PD-L1) antibody, and an anti-cytotoxic T-lymphocyte associated protein 4 (CTLA-4) antibody;
wherein the anti-programmed cell death-1 (PD-1) antibody is Pembrolizumab, the anti-programmed death-ligand antibody (PD-L1) is avelumab, and. the anti-cytotoxic T-lymphocyte associated protein 4 (CTLA-4) antibody is Ipilimumab.

39. (canceled)

40. The method of claim 1, wherein the V_{H} and the V_{L} are attached by a linker peptide, wherein the linker peptide comprises an amino acid sequence of SEQ ID NO: 4;
wherein the CD28 hinge region comprises an amino acid sequence of SEQ ID NO: 6;
wherein the CD28 transmembrane domain comprises an amino acid sequence of SEQ ID NO: 8;
wherein the CD28 costimulatory domain comprises an amino acid sequence of SEQ ID NO: 10;
wherein the CD3z activation domain comprises an amino acid sequence of SEQ ID NO: 11.

41-43. (canceled)

44. The method of claim 1, wherein the CAR further comprises a leader sequence, wherein the leader sequence comprises an amino acid sequence of SEO ID NO: 3.

45-47.

48. The method of claim 1, wherein the V_{H} and the V_{L} are attached by a linker peptide comprising an amino acid sequence of SEQ ID NO: 4 and the CD28 hinge region comprises the amino acid sequence of SEQ ID NO: 6;
wherein the CD28 transmembrane domain comprises an amino acid sequence of SEQ ID NO: 8;
wherein the CD28 costimulatory domain comprises an amino acid sequence of SEQ ID NO: 10;
wherein the CD3z activation domain comprises an amino acid sequence of SEQ ID NO: 11;
wherein the promoter is human elongation factor-1 alpha (EF-1α); and/or
wherein the scFv of a monoclonal antibody that binds to human EphA3, the hinge region, the transmembrane domain, and the intracellular signaling domain are fused in tandem.

49-54. (canceled)

55. The method of claim 1, wherein the CAR comprises amino acid sequence:
(SEQ ID NO: 13)
MALPVTALLLPLALLLHAARPQVQLVQSGAEVKKPGASVKVSCKASGYT
FTGYWMNWVRQAPGQGLEWMGDIYPGSGNTNYDEKFQGRVTMTRDTSIS
TAYMELSRLRSDDTAVYYCARGGYYEDFDSWGQGTTVTVSSGGGGSGGG
GSGGGGSDIQMTQSPSFLSASVGDRVTITCRASQGIISYLAWYQQKPEK
APKRLIYAASSLQSGVPSRFSGSGSGTEFTLTISSLQPEDFATYYCGQY
ANYPYTFGQGTKLEIKLEPKSCDKTHTCPPCPDPKFWVLVVVGGVLACY
SLLVTVAFIIFWVRSKRSRLLHSDYMNMTPRRPGPTRKHYQPYAPPRDF
AAYRSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPR.

56. The method of claim 22, wherein the V_{H} and the V_{L} are attached by a linker peptide, wherein the linker peptide comprises an amino acid sequence of SEQ ID NO: 4;
wherein the CD28 hinge region comprises an amino acid sequence of SEQ ID NO: 6;
wherein the CD28 transmembrane domain comprises an amino acid sequence of SEO ID NO: 8;
wherein the CD28 costimulatory domain comprises an amino acid sequence of SEQ ID NO: 10;
and/or wherein the CD3z activation domain comprises an amino acid sequence of SEQ ID NO: 11.

57-59. (canceled)

60. The method of claim 22, wherein the CAR further comprises a leader sequence, wherein the leader sequence comprises an amino acid sequence of SEQ ID NO: 3.

61-63. (canceled)

64. The method of claim 22, wherein the V_{H} and the V_{L} are attached by a linker peptide comprising an amino acid sequence of SEQ ID NO: 4 and the CD28 hinge region comprises the amino acid sequence of SEQ ID NO: 6;
wherein the CD28 transmembrane domain comprises an amino acid sequence of SEQ ID NO: 8;
wherein the CD28 costimulatory domain comprises an amino acid sequence of SEQ ID NO: 10;
wherein the CD3z activation domain comprises an amino acid sequence of SEQ ID NO: 11;
wherein the promoter is human elongation factor-1 alpha (EF-1α); and/or
wherein the scFv of a monoclonal antibody that binds to human EphA3, the hinge region, the transmembrane domain, and the intracellular signaling domain are fused in tandem.

65-70. (canceled)

71. The method of claim 22, wherein the CAR comprises amino acid sequence:
(SEQ ID NO: 13)
MALPVTALLLPLALLLHAARPQVQLVQSGAEVKKPGASVKVSCKASGYT
FTGYWMNWVRQAPGQGLEWMGDIYPGSGNTNYDEKFQGRVTMTRDTSIS
TAYMELSRLRSDDTAVYYCARGGYYEDFDSWGQGTTVTVSSGGGGSGGG
GSGGGGSDIQMTQSPSFLSASVGDRVTITCRASQGIISYLAWYQQKPEK
APKRLIYAASSLQSGVPSRFSGSGSGTEFTLTISSLQPEDFATYYCGQY
ANYPYTFGQGTKLEIKLEPKSCDKTHTCPPCPDPKFWVLVVVGGVLACY
SLLVTVAFIIFWVRSKRSRLLHSDYMNMTPRRPGPTRKHYQPYAPPRDF
AAYRSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPR.
